(19) United States
(12) Patent Application Publication
(10) Pub. No.: US 2015/0182479 A1
(43) Pub. Date: Jul. 2, 2015

(54) Method of delivering agmatine for medical treatment

(71) Applicants: Kenneth P. Glynn, Flemington, NJ (US); Craig P. Smith, Hillsborough, NJ (US)

(72) Inventors: Kenneth P. Glynn, Flemington, NJ (US); Craig P. Smith, Hillsborough, NJ (US)

(73) Assignee: TOPAZ VENTURES, INC.

(21) Appl. No.: 13/998,978

(22) Filed: Dec. 30, 2013

(51) Int. Cl. A61K 31/155; A61K 9/00; A61K 45/06

(52) U.S. Cl. CPC A61K 31/155; A61K 45/06; A61K 9/0043

(57) ABSTRACT

The present invention is directed to a method of delivering agmatine to a human for medicinal treatment, which includes: a.) providing a spray dispenser with an agmatine solution containing agmatine in a liquid carrier, the spray dispenser having a dispensing nozzle; and, b.) positioning the dispensing nozzle of the spray dispenser adjacent a nasal cavity of the human and spraying an effective amount of the agmatine solution into the nasal cavity so as to penetrate an olfactory area of the human.

### BACKGROUND OF INVENTION

a. Field of Invention

The invention relates generally to methods of delivering agmatine in liquid form to a patient by pressurized delivery to the nasal cavity to penetrate the olfactory region and deliver the agmatine rapidly to the brain, thereby reduing efficacy drop off before reaching the brain and decreasing dilutive effects that occur with other delivery mechanisms.

b. Description of Related Art

The following patents are representative of the field pertaining to the present invention:

U.S. Pat. No. 6,150,419 to Fairbanks et al describes a treatment and composition for neuropathic pain by administering an effective amount of agmatine.

U.S. Pat. No. 6,114,392 to Gilad et al describes the invention relates to the use of agmatine, in the treatment of acute neurotrauma (such as stroke) and degenerative disorders of the central and peripheral nervous system (such as dementia). The invention further provides novel compounds of general formula I (which are quinuclidine derivatives), formula II (which are norbomane derivatives), formula III (which are adamantane derivatives), and formula IV (which are phenothiazine derivatives): ##STR1## wherein R.sub.1, R.sub.2 and R.sub.3 are each independently hydrogen, hydroxy, substituted or unsubstituted C.sub.1-4 alkyl, substituted or unsubstituted C.sub.1-4 alkoxy, halogeno, amino, phenyl, or R.sub.4 NR.sub.5; R.sub.4 and R.sub.5 are each independently hydrogen, or (CH.sub.2)n--[NH(CH.sub.2)x]y--NHR.sub.6, or (CH.sub.2)n--[NH(CH.sub.2)x]y--NH—NHR.sub.6, or (CH.sub.2)n--[NH(CH.sub.2)x]y--(NR.sub.7.dbd.)CNHR.sub.6, or (CH.sub.2)n--[NH(CH.sub.2)x]y--NH(NR.sub.7.dbd.)CNHR.sub.6 wherein n is from 0-5, y is from 0-5 and each x is independently from 1-5; R.sub.6, and R.sub.7 are each independently hydrogen, hydroxy, substituted or unsubstituted C.sub.1-4 alkyl, substituted or unsubstituted C.sub.1-4 alkoxy, or halogeno; and pharmaceutically acceptable salts and optically active isomers thereof.

U.S. Pat. No. 5,677,349 to Gilad et al describes the invention relates to the use of agmatine, in the treatment of acute neurotrauma (such as stroke) and degenerative disorders of the central and peripheral nervous system (such as dementia).

United States Patent Application No. 2010/0172890 to Gilad et al describes the invention is dietary supplements, nutraceutical compositions, medical foods, and animal feeds that have cytoprotective (cell and tissue protection) and health promoting effects. The compositions contain a high dose range of agmatine and nutraceutical acceptable salts thereof as dietary fortification for providing effective long-term cytoprotection and affording for soft stool. The compositions may contain agmatine alone or in combination with other dietary ingredients having health promoting effects. The compositions can be prepared with dietary accepted excipients and compatible forms of carriers, including but not limited to, powders, tablets, capsules, controlled release carriers, lozenges and chewable preparations, liquid suspensions, suspensions in an edible supporting matrix or foodstuff and oral rehydration solutions, to enable consumption of said compositions.

United States Patent Application No. 2002/0065323 to Crooks et al describes Pharmaceutical preparations containing of agmatine, congeners, analogs or derivatives thereof for use in preventing or treating epilepsy, seizures and other electroconvulsive disorders are provided. Embodiments include administering an effective amount of agmatine, an agmatine analog or a pharmaceutically acceptable salt thereof to a human subject in need of treatment or prevention of epilepsy, seizure or other electroconvulsive disorder to treat, reduce, or prevent the disorder in the subject.

Notwithstanding the prior art, the present invention is neither taught nor rendered obvious thereby.

### SUMMARY OF INVENTION

The present invention is directed to a method of delivering agmatine to a human for medicinal treatment, which includes: a.) providing a spray dispenser with an agmatine solution containing agmatine in a liquid carrier, the spray dispenser having a dispensing nozzle; and, b.) positioning the dispensing nozzle of the spray dispenser adjacent a nasal cavity of the human and spraying an effective amount of the agmatine solution into the nasal cavity so as to penetrate an olfactory area of the human.

In some preferred embodiments of the present invention method of delivering agmatine to a human for medicinal treatment, the liquid carrier is water. Other non-active or active carriers could be used, such as juice-water solutions, saline, water with small amounts of honey, or other natural additive.

In some preferred embodiments of the present invention method of delivering agmatine to a human for medicinal treatment, the medicinal treatment is for a stroke ailment.

In some preferred embodiments of the present invention method of delivering agmatine to a human for medicinal treatment, the medicinal treatment is preventative medical treatment for a stroke ailment taken before ailment occurs.

In some preferred embodiments of the present invention method of delivering agmatine to a human for medicinal treatment, the medicinal treatment for spinal cord injury.

In some preferred embodiments of the present invention method of delivering agmatine to a human for medicinal treatment, the medicinal treatment is preventative medical treatment taken for spinal cord injury before the ailment occurs.

In some preferred embodiments of the present invention method of delivering agmatine to a human for medicinal treatment, the medicinal treatment is an adjunct treatment with opiate treatment for pain.

In some preferred embodiments of the present invention method of delivering agmatine to a human for medicinal treatment, the medicinal treatment is an adjunct treatment with cannabinoid treatment for pain.

In some preferred embodiments of the present invention method of delivering agmatine to a human for medicinal treatment, the medicinal treatment is for depression ailments.

In some preferred embodiments of the present invention method of delivering agmatine to a human for medicinal treatment, the medicinal treatment is for traumatic brain injury.

In some preferred embodiments of the present invention method of delivering agmatine to a human for medicinal treatment, the medicinal treatment is for traumatic brain injury and is preventative medical treatment taken before a traumatic brain injury occurs.

In some preferred embodiments of the present invention method of delivering agmatine to a human for medicinal treatment, the medicinal treatment is for traumatic brain injury and is curative medical treatment taken after a traumatic brain injury occurs.

In some preferred embodiments of the present invention method of delivering agmatine to a human for medicinal treatment, the medicinal treatment is for traumatic brain injury and is preventative medical treatment taken before a traumatic brain injury occurs and is also curative medical treatment taken after a traumatic brain injury occurs.

In some preferred embodiments of the present invention method of delivering agmatine to a human for medicinal treatment, the spray dispenser is selected from the group consisting of a pressurized spray dispenser and a manual pumped dispenser.

In some preferred embodiments of the present invention method of delivering agmatine to a human for medicinal treatment, the spray dispenser dispenses the agmatine solution at a rate of 0.05 to 4.0 cc/sec.

In some preferred embodiments of the present invention method of delivering agmatine to a human for medicinal treatment, the agmatine solution contains about 2 to about 200 mg of agmatine per ml of liquid carrier. In some of the more preferred embodiments of the present invention method of delivering agmatine to a human for medicinal treatment, the agmatine solution contains about 4 to about 40 mg of agmatine per ml of liquid carrier. In some of the most preferred embodiments of the present invention method of delivering agmatine to a human for medicinal treatment, the agmatine solution contains about 5 to about 15 mg of agmatine per ml of liquid carrier.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 is a block diagram of embodiments of the present invention agmatine nasal delivery methods and treatments, with and without one or more additional active components;

All of the following Figures are described as agmatine solutions, it being understood that this means with and without at least one additional active component;

FIG. 2 is a block diagram showing ailments treated by various embodiments of the present invention agmatine nasal delivery methods and treatments;

FIG. 3 is a block diagram showing durations for medicinal non-inhaled dosage in some preferred embodiments of the present invention agmatine nasal delivery methods and treatments;

FIG. 4 is a block diagram of another embodiment of the present invention agmatine nasal delivery methods and treatments, showing the additional step of repeating the initial steps;

FIG. 5 is a block diagram showing flow rates in some additional preferred embodiments of the present invention agmatine nasal delivery methods and treatments;

FIG. 6 is a block diagram showing the concentration of agmatine present in the liquid carrier;

FIG. 7 illustrates a block diagram showing monodose and multidose dispensers that may be used in the present invention methods;

FIG. 8 illustrates a front partially cut view of one embodiment of a present invention nasal treatment delivery device with a pressure release mechanism;

FIG. 9 illustrates a view of one embodiment of a present invention nasal treatment delivery device that is a squeeze to release device;

FIG. 10 shows a front partially cut view of a present invention nasal treatment delivery device with a piercing channel, with the device being held in a hand using two fingers and a thumb to activate release of the medicinal treatment;

FIGS. 11, 12 and 13 illustrate front partially cut views of one embodiment of a present invention nasal treatment delivery device with a frangible internal medicine capsule that may be used for a monodose or multidose using replacement cartridges. The three Figures show the device in different stages of use; and,

FIGS. 14 and 15 show alternative types of dosage dispenser heads that may be used in present invention devices, one showing multiple release ports and the other showing multiple release ports with a soft contact sheath.

### DETAILED DESCRIPTION OF THE EMBODIMENTS

Referring now in detail to the drawings wherein like reference numerals designate corresponding parts throughout the several views, various embodiments of the present invention are shown. Many uses have been discovered for agmatine over the past century, and, notwithstanding its apparent benefits, most entities have abandoned its use for their particular application for one or more of numerous reasons: (a) dosages was be so high to be effective that it would be impractical; (b) the level efficacy was insufficient to replace alternative existing medicines; (c) the rapid dissipation levels made usage impractical; at required dosages combined with need frequencies, it would not be cost effective.

The net result is that after almost 100 years of research and development on agmatine, no products or teachings exist beyond homeopathic pills for human consumption. The inventors herein, however, identified the functionary aspects of agmatine as primarily, and in some cases, exclusively, brain related. Thus, it was observed that delivery times from injections, exodermic, pill or other delivery to the brain caused both dissipation and lost efficacy. Blasting into the nasal area with pressurized spray for delivery has been found to penetrate the olfactory region and affect rapid delivery to the brain, allowing most of the agmatine to arrive where it is active rather than relying upon the blood stream where dissipation, delay and diffusion occur before only a small amount reaches the brain.

Many uses have been tested for agmatine, and the results show effectiveness, but not sufficient to substute agmatine for existing medications. However, with the present invention rapid, local delivery method, agamatine becomes a viable medicine for various ailments that are treated at the brain regions. Some of the established uses are now described:

(1.) Neuroprotective Agent.

Jinn-Rung Kuo, MD, Chong-Jeh Lo, MD, Ching-Ping Chang, PhD, Kao-Chang Lin, MD, Mao-Tsun Lin, DDS, PhD, and Chung-Ching Chio, MD in a paper titled “Agmatine-Promoted Angiogenesis, Neurogenesis, and Inhibition of Gliosis-Reduced Traumatic Brain Injury in Rats” (J Trauma. 2011; 71: E87-E93) established that agmatine is an effective neuprotective agent for Traumatic Brain Injury (hereinafter “TBI”). The study was to test whether inhibition of gliosis, angiogenesis, and neurogenesis attenuating TBI could be agmatine stimulated. Traumatic brain injury (TBI), the second largest killer in the world, and is a major burden for the societies in terms of cost, suffering, and disability in all developed countries. According to prior studies Kuo J R, Lo C J, Chio C C, Chang C P, Lin M T. “Resuscitation From Experimental Traumatic Brain Injury By Agmatine Therapy” Resuscitation. 2007; 75:506-514, neuronal loss after TBI is focal and diffuse. Focal neuronal loss occurred by necrotic and apoptotic mechanisms. See also, Gilad G M, Salame K, Rabey J M, Gilad V H. “Agmatine Treatment Is Neuroprotective In Rodent Brain Injury Models” Life Sci. 1996; S8:PL41-PL46. Additionally, apoptotic neurons were observed in the hippocampus. In response to TBI, as was observed in rodents, generating new neurons and inhibiting gliosis (or scar formation) might be responsible for recovery of TBI. Agmatine, an endogenous polyamine formed by decarboxylation of L-arginine, was reported to have neuroprotective effect in TBI. However, it is unknown whether agmatine-stimulated angiogenesis, neurogenesis, and inhibition of gliosis (or scar formation) attenuates TBI. Methods: Anesthetized rats were randomly assigned to sham-operated group, TBI rats treated with saline (1 mL/kg, intraperitoneally), or TBI rats treated with agmatine (50 mg/kg, intraperitoneally). Saline or agmatine was injected 5 minutes after TBI and again once daily for the next 3 postoperative days. Results: Agmatine therapy in rats significantly attenuated TBI-induced motor function deficits (62° vs. 52° maximal angle) and cerebral infarction (88 mm3 vs. 216 mm3), significantly reduced TBI-induced neuronal (9 NeuN-TUNEL double positive cells vs. 60 NeuN-TUNEL double positive cells) and glial (2 GFAP-TUNEL double positive cells vs. 20 GFAP-TUNEL double positive cells) apoptosis (increased TUNEL-positive and caspase-3-positive cells), neuronal loss (82 NeuN-positive cells vs. 60 NeuN-positive cells), gliosis (35 GFAP-positive cells vs. 72 GFAP-positive cells; 60 Iba1-positive cells vs. 90 Iba1-positive cells), and neurotoxicity (30 n-NOS-positive cells vs. 90 n-NOS-positive cells; 35 3-NT-positive cells vs. 90 3-NT-positive cells), and significantly promoted angiogenesis (3 BrdU/endothelial cells vs. 0.5 BrdU/endothelial cells; 50 vascular endothelial growth factor positive cells vs. 20 vascular endothelial growth factor-positive cells) and neurogenesis (27 BrdU/NeuN positive cells vs. 15 BrdU/NeuN positive cells).

Resultantly, agmatine therapy, to some degree, attenuated TBI in rats via promoting angiogenesis, neurogenesis, and inhibition of gliosis. The authors' results are stated by them as follows:

Agmatine Improved Motor Dysfunction During TBI:

Seven days after TBI, behavioral tests revealed that vehicle-treated TBI rats had significantly lower performance in motor function test than they were for sham-operated controls. The TBI-induced motor dysfunction could be significantly reduced by agmatine therapy.

Agmatine Decreased Infarct Volume During TBI:

The TTC-stained sections at 7 days after TBI showed a significant increase in the infracted area of the vehicle-treated TBI as compared with those of sham TBI controls. The TBI-induced infarction volume was significantly decreased by agmatine treatment.

Agmatine Decreased Neuronal Loss, Astrogliosis, and Microgliosis During TBI:

As evaluated at 7 days after TBI, the vehicle-treated TBI rats had lower numbers of NeuN-positive cells but higher number of both GFAP-positive cells and Iba1-positive cells in the ischemic cortex compared with those of sham TBI controls.

Agmatine Decreased Both Neuronal and Glial Apoptosis During TBI:

At 7 days after TBI, it was found that vehicle-treated TBI rats had significantly higher numbers of NeuN plus TUNEL positive cells, GFAP plus TUNEL positive cells, and caspase-3-positive cells in the ischemic cortex compared with those of sham TBI controls. Their figures also showed that the increased numbers of NeuN plus TUNEL-positive cells, GFAP plus TUNEL-positive cells, and caspase-3-positive cells in the ischemic cortex after TBI were significantly decreased by agmatine therapy.

Agmatine Promoted Neurogenesis During TBI:

At 7 days after TBI, the vehicle-treated TBI rats had significantly higher numbers of BrdU/NeuN double positive cells and GDNF-positive in the ischemic cortex compared with those of sham TBI controls. Again, the increased numbers of both BrdU/NeuN doublepositive cells and GDNF-positive cells in the ischemic cortex were significantly decreased by agmatine therapy.

Agmatine Promoted Angiogenesis During TBI

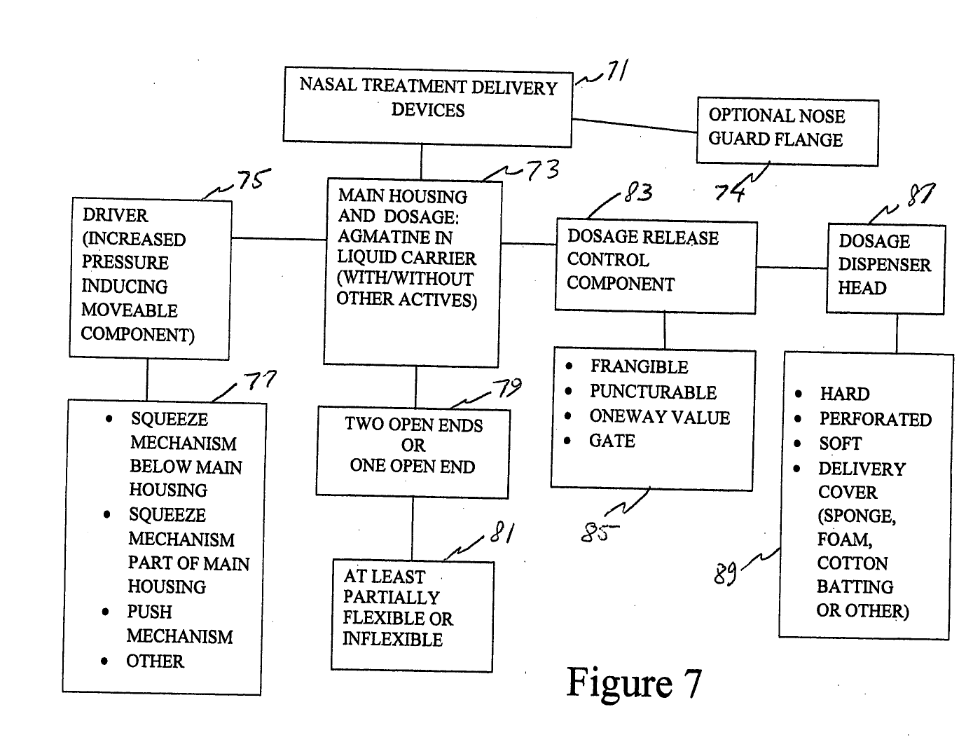

As evaluated at 7 days after TBI, the number of both BrdU-positive endothelial cells (FIG. 7, A) and VEGF-positive cells (FIG. 7, B) in the ischemic cortex of vehicle-treated TBI rats were significantly higher than those of sham TBI rats. Again, the increased numbers of both BrdU-positive endothelial cells and VEGF-positive cells in the ischemic cortex after TBI were further significantly increased by agmatine therapy (FIGS. 7, A and B).

Agmatine Decreased Both n-NOS and 3-NT Expression During TBI

As revealed at 7 days after TBI, the numbers of both n-NOS-positive cells (FIGS. 8, A) and 3-NT-positive cells (FIG. 8, B) in the ischemic cortex of vehicle-treated TBI rats were significantly higher than those of sham TBI rats. Again, the increased numbers of both n-NOS-positive cells and 3-NTpositive cells in the ischemic cortex after TBI were significantly decreased by agmatine therapy (FIGS. 8, A and B).

They further concluded that their findings demonstrated that agmatine might improve motor dysfunction and cerebral infarction and apoptosis that occurred during TBI by stimulating production of GDNF in the ischemia brain. As shown in the present study, agmatine therapy increased the amounts of both BrdU-positive endothelial cells and VEGF-positive cells in the injured brain, attenuated cerebral infarction and apoptosis, and restored normal motor function in a rat TBI model. A rich vascular environment, along with generation of VEGF, might enhance subsequent angiogenesis and neurogenesis. A more recent report also showed that systemic delivery of Premarin, a soluble estrogen sulfate, attenuated TBI-induced cerebral infarction and apoptosis by increasing the amounts of both VEGF positive cells and BrdU-positive endothelial cells in the injured brain. Thus, agmatine might improve motor outcome during TBI by enhancing neovessel formation and accelerating endogenous neurogenesis. Decisive evidence indicated that nitric oxide overproduction from neuronal nitric oxide synthase impaired brain tissue. Poor neurologic outcome was also associated with increased levels of nitrotyrosine in the cerebrospinal fluid in human TBI. 3-NT was shown to be involved in the induction of both motor neuron apoptosis in vitro and mitochondrial oxidative damage and dysfunction in a mouse model of focal TBI. Agmatine was believed to be synthesized predominantly by the astroglia cells, then released and taken up into neurons by active transport.29, 30 Agmatine also acted as an irreversible inactivator of n-NOS. In the current studies, TBI-induced neuronal and glial apoptosis, and overexpression of both n-NOS and 3-NT in the ischemia brain could be significantly reduced by agmatine treatment. Together, these results suggested that agmatine might cause attenuation of neuronal and glial apoptosis by reducing overexpression of both n-NOS and 3-NT in the ischemic brain during TBI.

In this study, 7 days after TBI, gliosis (evidenced by increased numbers of both GFAP-positive cells and Iba1-positive cells) and neuronal and glial apoptosis (evidenced by increased numbers of NeuN plus TUNELpositive cells, GFAP plus TUNEL-positive cells, and Iba1 plus TUNEL-positive cells) in the ischemia cortex, could be significantly reduced by agmatine therapy. The reduction of both gliosis and neuronal and glial apoptosis in agmatine treated TBI animals was paralleled by the reduced infarct volume and near normal motor function. These results indicated the agmatine might protect against the delayed infarct expansion caused by activated astrocytes during TBI. Astrocytes are believed to be responsible for most glutamate uptake in synaptic and monosynaptic areas and consequent are the major regulators of glutamate homeostasis. Microglia may secrete cytokines, which can impair glutamate uptake. These observations indicate that agmatine may protect against TBI-induced apoptosis via reducing glutamatemediated glial injury.

(2) Resuscitation from Experimental Traumatic Brain Injury by Agmatine Therapy

A group of Tawianese experts, Jinn-Rung Kuoa, Chong-Jeh Loa, Chung-Ching Chiob, Ching-Ping Changc, and Mao-Tsun Lind, in a paper by the above title, performed extensive research on agmatine therapy for traumatic brain injury (TBI). It has been observed that both nitric oxide and glutamate contribute to ischaemic brain injury. Agmatine inhibits all isoforms of nitric oxide synthase and blocks N-methyl-Daspartate receptors. In this study, they gave agmatine intraperitoneally and assessed its effect on fluid percussion brain injury in rats. Anaesthetised rats, immediately after the onset of fluid percussion traumatic brain injury (TBI), were divided into two major groups and given the vehicle solution (1 mL/kg) or agmatine (50 mg/kg) intraperitoneally. Mean arterial pressure, intracranial pressure, cerebral perfusion pressure, and levels of glutamate, nitric oxide, lactate/pyruvate ratio, and glycerol in hippocampus were monitored continuously within 120 min after TBI. The weight loss was determined by the difference between the first and third day of body weight after TBI. The maximal grip angle in an inclined plane was measured to determine motor performance whereas the percent of maximal possible effect was used to measure blockade of proprioception. The triphenyltetrazolium chloride staining procedures were used for cerebral infarction assay. Compared to those of the sham-operated controls, the animals with TBI had higher values of extracellular levels of glutamate, nitric oxide, lactate-to-pyruvate ratio, and glycerol in hippocampus and intracranial pressure, but lower values of cerebral perfusion pressure. Agmatine administered immediately after TBI significantly attenuated the TBI-induced increased hippocampal levels of glutamate, nitric oxide, lactate-topyruvate ratio, and glycerol, intracranial hypertension, and cerebral hypoperfusion. In addition, the TBI-induced cerebral infarction, motor and proprioception deficits, and body weight loss evaluated 3 days after TBI were significantly attenuated by agmatine therapy. The present data indicate that agmatine may attenuate TBI by reducing the excessive accumulation of both glutamate and nitric oxide in the brain.

Results:

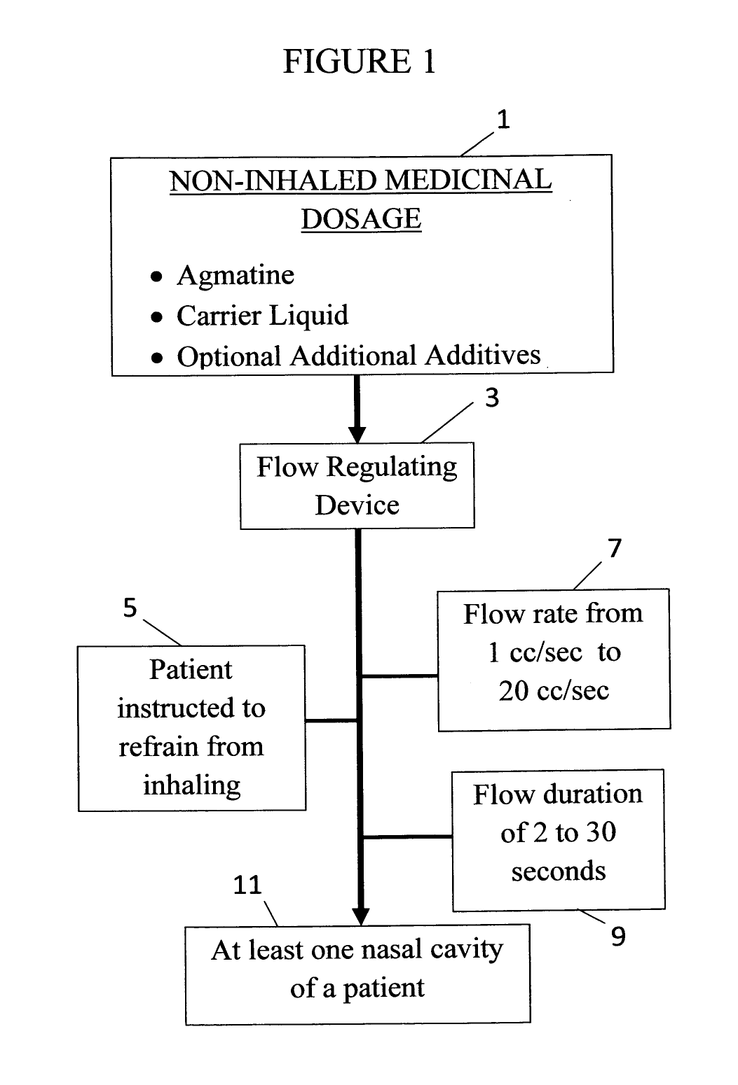

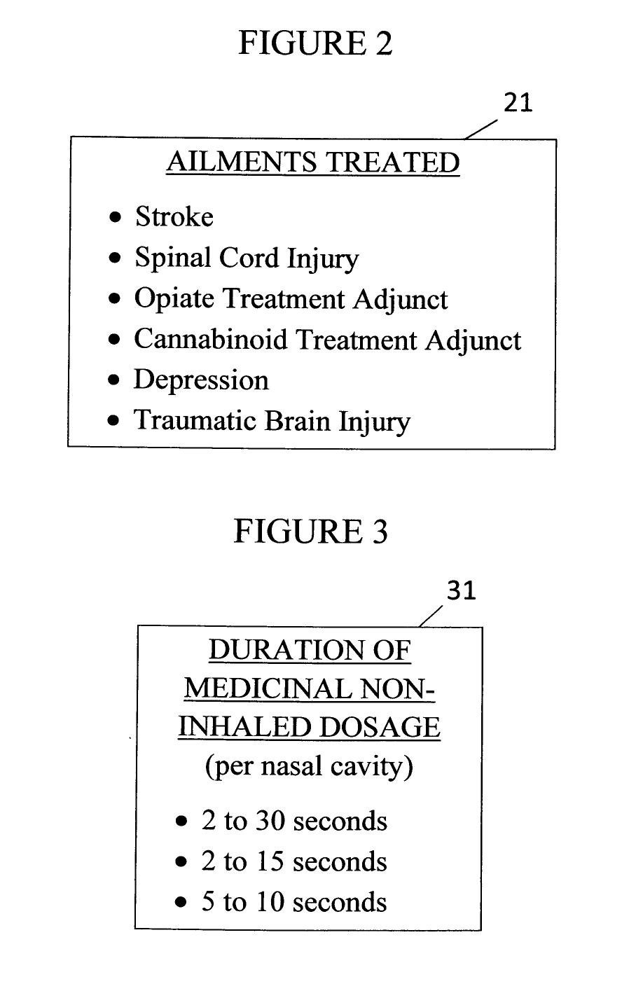

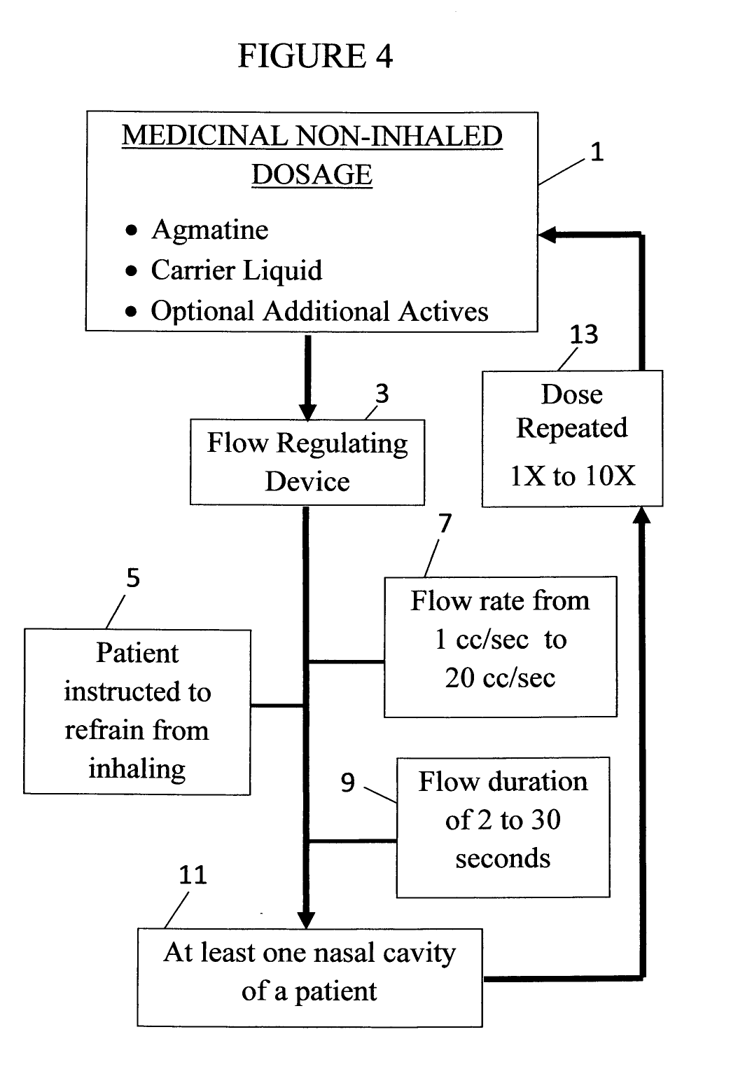

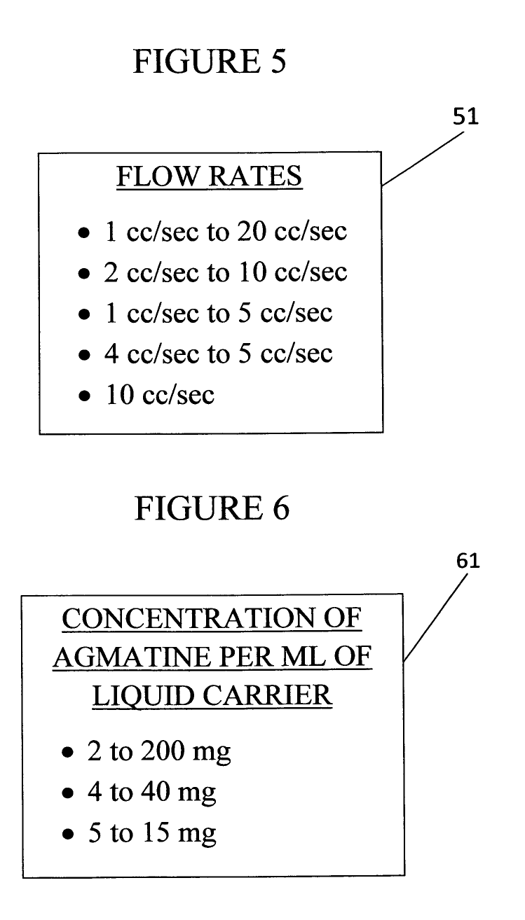

Their FIGS. 1 and 2 depict the effects of FPI on several cerebrovascular variables as well as extracellular levels of glycerol, glutamate, lactate/pyruvate ratio, and NO2—in hippocampus in rats treated with vehicle solution, and in rats treated with agmatine. In vehicle-treated FPI group, the HR, ICP, and hippocampal levels of glycerol, glutamate, lactate/pyruvate ratio, and NO2—were all significantly higher at 10-120 min after the start of FPI than they were for shamoperated controls. In contrast, the values for CPP were significantly lower than those of shamoperated controls. Resuscitation with agmatine immediately after FPI significantly attenuated the FPI-induced intracranial hypertension, cerebral hypoperfusion and overproduction of cellular ischemia and injury markers in hippocampus. The basal levels of cerebrovascular parameters and ischaemia and injury markers measured in shamoperated rats treated with agmatine (50 mg/kg,i.p.) were indistinguishable from those of shamoperated rats received no treatment (data notshown). Their FIG. 3 shows that FPI rats treated with vehicle solution immediately after injury have higher amounts of weight loss compared to those of shamoperated controls. The weight loss denotes the difference in body weight between the first and third day after FPI. However, agmatine therapy (50 mg/kg, i.p.) immediately after FPI significantly reversed the FPI-induced weight loss. As compared with those of the sham-operated control rats, the maximal angle animals treated with vehicle solution could cling to an inclined plane significantly decreased 72 h after FPI (as shown in their FIG. 4). However, the FPI-induced reduction in maximum grip angle measured 72 h after FPI was reversed significantly by agmatine therapy (50 mg/kg, i.p.) (P<0.05). The percent of MPE of proprioception blockade 72 h after FPI increased significantly in vehicle solution-treated FPI animals compared to those of sham-operated controls (their FIG. 5). Again, the % of MPE of proprioception blockade obtained 72 h after FPI was reversed significantly by agmatine administration( ) (P<0.05). Triphenyltetrazolium chloride staining revealed that the marked increase in cerebral infarction in FPI rats treated with vehicle solution (their FIG. 6C). Compared to those of sham-operated controls, FPI induced a significant increase in cerebral infarction volume (their FIG. 6A) and incidence (their FIG. 6B) in rats treated with vehicle solution. Again, the FPI-induced cerebral infarction in terms of both volume and % incidence was reduced significantly by treatment with agmatine (50 mg/kg, i.p.) (P<0.05).

The resulting data indicated that agmatine may attenuate traumatic brain injury by reducing the excessive accumulation of both glutamate and nitric oxide in brain.

(3) Agmatine Enhances Cannabinoid Action in the Hot-Plate Assay of Thermal Nociception

Saniya Aggarwal, Behnam Shavalian, Esther Kim, and Scott M. Rawls, all Department of Pharmaceutical Sciences, Temple University Health Sciences Center, Philadelphia, Pa., USA and Department of Pharmacology, Temple University Health Sciences Center, Philadelphia, Pa., USA And Scott Rawls also from the Center for Substance Abuse Research, Temple University, Philadelphia, Pa., USA studied the interrelationships between agmatine and cannabinoid, and reported in a paper titled as above, available online 16 Jun. 2009, the results: Agmatine-cannabinoid interactions are supported by the close association between cannabinoid CB1 receptors and agmatine immunoreactive neurons and evidence that shared brain mechanisms underlie the pharmacological effects of agmatine and cannabinoid agonists. The present study used the hot-plate assay of thermal nociception to determine if agmatine alters cannabinoid action through activation of imidazoline sites and/or alpha2-adrenoceptors. Administration of WIN 55212-2 (1, 2 or 3 mg/kg, i.p.) or CP55,940 (1, 2 or 3 mg/kg, i.p.) forms of cannabinoid, increased hot-plate response latency. Agmatine (50 or 100 mg/kg, i.p.) was ineffective. Administration of agmatine (50 mg/kg, i.p.) with WIN 55212-2 (1, 2 or 3 mg/kg, i.p.) or CP55,940 (1, 2 or 3 mg/kg, i.p.) produced response-latency enhancement. Regression analysis indicated that agmatine increased the potency of WIN 55212-2 and CP55,940 by 3- and 4.4-fold, respectively, indicating synergy for both drug interactions. Idazoxan, a mixed imidazoline site/alpha2-adrenoceptor antagonist, but not yohimbine (5 mg/kg, i.p.), a selective alphia2-adrenoceptor antagonist, blocked response-latency enhancement produced by a combination of WIN 55212-2 (2 mg/kg) and agmatine. Response-latency enhancement produced by WIN 55212-2 (2 mg/kg) was blocked by SR 141716A (5 mg/kg, i.p.), a cannabinoid CB1 receptor antagonist; attenuated by idazoxan (2 and 5 mg/kg); and not affected by yohimbine (5 mg/kg). These results demonstrate a synergistic interaction between agmatine and cannabinoid agonists and suggest that agmatine administration enhances cannabinoid action in vivo.

In addition to activating imidazoline sites, agmatine displays affinity for alpha2-adrenoceptors and antagonizes glutamatergic NMDA receptors. Agmatine also inhibits neuronal nitric oxide synthase and downregulates inducible nitric oxide synthase. In the mammalian brain, agmatine is synthesized by the enzyme arginine decarboxylase and degraded by the enzymeagmatinase. Central effects of agmatine include aweak analgesic action, anti-depressant like effects, reduction of seizure-evoked glutamate levels in the frontal cortex, attenuation of neuropathic pain, anti-convulsant effects, improvement of locomotor function following spinal cord injury and blockade of stress- and bacterial endotoxin-evoked hyperthermia. Agmatine is well known for its interaction with mu opioid receptors. Results reveal that agmatine administration blocks all symptoms of morphine withdrawal, enhances acutemorphine analgesia and prevents tolerance to morphine analgesia Despite the well-documented ability of agmatine to modulate opioid function, its role in cannabinoid function is not yet clear. Prior work suggests that agmatine enhances the hypothermic effect of a cannabinoid agonist, but it is not known if additional cannabinoid-induced actions are modulated by agmatine In their study, they investigated the effect of exogenous agmatine on cannabinoid action in the hot-plate assay of thermal nociception and determined whether imidazoline sites and alpha2-adrenoceptors contributed to the agmatine-cannabinoid interaction.

WIN 55212-2 ([4,5-dihydro-2-methyl-4(4-morpholinylmethyl)-1-(1-naphthalenylcarbonyl)-6H-pyrrolo[3,2,1ij]quinolin-6-one]), CP55,940((−)-cis 3-(2-hydroxy-4-(1,1-dimethylheptyl)phenyl)-trans-4-(3-hydroxypropyl) cyclohexanol, arachidonylethanolamide (anandamide), agmatine sulfate and yohimbine were utilized.

Results:

Effect of agmatine on hot-plate response latency: The effect of agmatine (50 and 100 mg/kg) by itself on the hot-plate response latency is presented in FIG. 1. A two-way ANOVA on the individual response latencies revealed that there was not a significant drug interaction (F2, 20=3.054, P<0.05), time interaction (F3, 60=2.050, P<0.05), or drug time interaction (F6, 60=0.3097, P<0.05).

Results:

Effect of agmatine and cannabinoid co-administration on hot-plate response latency: The effect of a fixed, inactive dose of agmatine (50 mg/kg) on the increase in response latency caused by progressively increasing doses of WIN 5521-2 (1, 2 and 3 mg/kg) is displayed in FIG. 2. This dose of agmatine (50 mg/kg), which by itself did not alter the response latency (FIG. 1), enhanced the increase in response latency caused by each dose of WIN 55212-2 (1, 2 and 3 mg/kg) (P<0.05, Student's t-test, FIG. 2a-c). Using the data in FIG. 2a-c, we compared the dose-response relation of the active agent, WIN 55212-2, and the dose-response relation of WIN 5212-2 in combination with the inactive agent, agmatine (FIG. 2d). These two dose-response data sets, using the effect level of response latency 30 min post-administration, were used to construct regression lines (effect on log dose) in FIG. 2d. Regression analysis revealed a pronounced leftward shift in the combination's regression line (FIG. 2d). Because the lines did not differ significantly in slope (P<0.05), this shift was expressed in terms of relative potency (R), which is defined as the ratio of theamount of each drug required to produce the same effect (i.e., the ratio of ED50 values for the active drug alone and the active drug in combination with the inactive agent). R, computed with the assistance of PharmTools Pro (TheMcCary Group, Elkins Park, Pa.), was found to be 2.72, with 95% confidence limits (1.609 to 7.367). This value of R, significantly greater than unity, indicates synergism for the interaction between WIN 55212-2 and agmatine. Agmatine (50 mg/kg) had a similar effect on CP55,940 (1 or 2 mg/kg) (Pb0.05, FIG. 3a-b). Regression analysis comparing the dose-response relation of CP55,940 and dose-response relation of CP55,940 in combination with agmatine revealed a pronounced leftward shift in the combination's regression line (FIG. 3d). The regression lines did not differ significantly in slope (P<0.05) and R was found to be 4.39, with 95% confidence limits of 3.192 to 5.596, indicating the interaction was synergistic. The increase in response latency produced by the highest dose (3 mg/kg) of CP55,940 was also enhanced in the presence of agmatine, but the effect did reach statistical significance (P<0.05) (FIG. 3c). Agmatine (50 mg/kg) did not significantly enhance the increase in response latency following anandamide (3 or 7.5 mg/kg, i.p.) administration (P<0.05) (FIG. 4).

(4) Augmentation of Morphine and Oxycodone Analgesia

Shaifali Bhalla, Vaide Rapolaviciute and Anil Gulati all of Midwestern University, Downers Grove, Ill. 60515, United States published a paper (online 27 Nov. 2010) titled “Determination of a2-adrenoceptor and imidazoline receptor involvement in augmentation of morphine and oxycodone analgesia by agmatine and BMS 182874”. Prior studies had demonstrated that clonidine (α2-adrenoceptor and imidazoline receptor agonist) and BMS 182874 (endothelin ETA receptor antagonist) potentiate morphine and oxycodone analgesia. Agmatine, an endogenous clonidine-like substance, enhances morphine analgesia. However, its effect on oxycodone analgesia and its interaction with endothelin ETA receptor antagonists were not known. Their study was performed to determine the effect of agmatine on morphine and oxycodone analgesia and the involvement of α2-adrenoceptors, imidazoline receptors, opioid receptors, and endothelin receptors. Antinociception at various time intervals was determined by the tail-flick latency method in mice. Agmatine produced dose-dependent increase in tail-flick latency, while BMS 182874 did not produce any change over the 360-min observation period. Agmatine significantly potentiated morphine as well as oxycodone analgesia which was not altered by BMS 182874. BMS 182874 pretreatment did not increase the analgesic effect produced by agmatine alone. Agmatine-induced potentiation of morphine and oxycodone analgesia was blocked by idazoxan (imidazoline receptor/α2-adrenoceptor antagonist) and yohimbine (α2-adrenoceptor antagonist). BMS 182874-induced potentiation of morphine or oxycodone analgesia was not affected by yohimbine. However, idazoxan blocked BMS 182874-induced potentiation of oxycodone but not morphine analgesia. This is the first report demonstrating that agmatine potentiates not only morphine but also oxycodone analgesia in mice. Potentiation of morphine and oxycodone analgesia by agmatine appears to involve α2-adrenoceptors, imidazoline receptors, and opioid receptors. In addition, imidazoline receptors may be involved in BMS 182874-induced potentiation of oxycodone but notmorphine analgesia. They concluded that agmatine may be used as an adjuvant in opiate analgesia.

Opioids are one of the most potent classes of analgesics to treat severe acute aswell as chronic pain. They are the favored drug of choice in clinical situations because of their high analgesic efficacy, however, a number of side effects develop after their prolonged use. The most serious adverse effects are sedation, tolerance, drug dependence, hyperalgesia, constipation, respiratory depression, and miosis. Mechanisms involved in these negative outcomes are very complex and involve opioid and non-opioid systems. Non-opioid systems like gamma butyric acid (GABA), dopamine, nitric oxide, N-methyl-D-aspartate (NMDA), and glutamate play important roles in the development of adverse effects mentioned.

An endogenous clonidine-like substance, agmatine, enhances morphine induced analgesia when given systemically. Agmatine by itself is a weak analgesic, but studies have shown that it enhances antinociceptive action of morphine and inhibits the development of tolerance and dependence on opioids as well. Endothelin-1 causes nociception and hyperalgesia by binding to endothelin ETA receptors localized on nociceptors.

All anesthetic and surgical procedures were in compliance with the guidelines established by the IACUC at Midwestern University.

Results:

FIG. 1. Effect of agmatine on three different doses of morphine (2 mg/kg, 4 mg/kg, and 8 mg/kg, s.c.). Agmatine (10 mg/kg, i.p.) or vehicle (10 ml/kg, i.p.) was administered 30 min before morphine (2, 4, or 8 mg/kg, s.c.) treatment. Tail flick latency responses were measured at various time intervals. Antinociceptive response was determined by the tail-flick latency method. Application of thermal radiation (by focused light) to the tail of the animal provoked the withdrawal of the tail by a brief vigorous movement. The withdrawal time was recorded as the tail-flick latency by using an. Tailflick latencies to thermal stimulation were determined at baseline (before any drug administration) and at 30, 60, 90, 120, 180, 240, 300, and 360 min after injection of saline, morphine, or oxycodone. A cutoff time of 10 s was used to prevent damage to the tail.

Results:

FIG. 2. Shows the effect of agmatine on three different doses of oxycodone (5 mg/kg, 15 mg/kg, and 45 mg/kg, s.c.). Agmatine (10 mg/kg, i.p.) or vehicle (10 ml/kg, i.p.) was administered 30 min before oxycodone (5, 15, or 45 mg/kg, s.c.) treatment. Tail flick latency responses were measured at various time intervals.

Conclusion:

The results confirmed previous findings that endothelin ETA receptor antagonist BMS 182874 potentiates morphine as well as oxycodone analgesia in mice. This is the first report showing that agmatine potentiates oxycodone induced analgesia in mice. The study also provides evidence that α2-adrenoceptors are involved in the potentiation of morphine and oxycodone analgesia by agmatine, but not in BMS 182874 induced potentiation of morphine and oxycodone analgesia.

(4) Modulation of Opioid Analgesia by Agmatine

Yuri Kolesnikov, Subash Jain and Gavril W. Pasternak published a paper by the above title showing agmatine modulating opiod analgesia. Administered alone, agmatine at doses of 0.1 or 10 mg/kg is without effect in the mouse tailflick assay. However, agmatine enhances morphine analgesia in a dose-dependent manner, shifting morphine's EDsO over 5-fold. A far greater effect is observed when morphine is given intrathecally (9-fold shift) than after intracerebroventricular administration (2-fold). In contrast to the potentiation of morphine analgesia, agmatine (10 mg/kg) has no effect on morphine's inhibition of gastrointestinal transit. Opioid receptor-mediated analgesia also is potentiated by agmatine, but Kl-receptor-mediated (U50,488H; trans-3,4-dichloro-N-methyl-N-[2-(1-pyrrolidinyl)cyclohexyi]benzeneacetemide) and K3-opioid receptor-mediated (naloxone benzoylhydrazone) analgesia is not significantly enhanced by any dose of agmatine tested in this acute model. In chronic studies, agmatine at a low dose (0.1 mg/kg) which does not affect morphine analgesia acutely prevents tolerance following chronic morphine dosing for 10 days. A higher agmatine dose (10 mg/kg) has a similar effect. Agmatine also blocks tolerance to the opioid receptor ligand [D-Pen2,D-PenS]enkephalin given intrathecally, but not to the K3-opioid receptor agonist naloxone benzoylhydrazone. Despite its inactivity on Kl-opioid analgesia in the acute model, agmatine prevents Kt-opioid receptor-mediated tolerance. These studies demonstrate the dramatic interactions between agmatine and opioid analgesia and tolerance.

(5) A Biphasic Opioid Function Modulator: Agmatine

Su Rui-Bin, LI Jin and QIN Bo-Yi describes this agamtine attribute in a paper by the above title. They report that some agents that are not able to interact with opioid receptors play an important role in regulating the pharmacological actions of opioids. Especially, some of them show biphasic modulation on opioid functions, which enhance opioid analgesia, but inhibit tolerance to and substance dependence on opioids. They call these agents which do not interact with opioid receptors, but do have biphasic modulation on opioid functions as biphasic opioid function modulator (BOFM). Mainly based on their results, agmatine is a typical BOFM. Agmatine itself was a weak analgesic which enhanced analgesic action of morphine and inhibited tolerance to and dependence on opioid. The main mechanisms of agmatine were related to inhibition of the adaptation of opioid receptor signal transduction induced by chronic treatment of opioid.

(6) A Preliminary Histopathological Study of the Effect of Agmatine on Diffuse Brain Injury in Rats

A group of diverse Turkish researchers, Goksin Sengul, Erhan Takci, Umit Ali Malcok, Ali Akar, Fazli Erdogan, Hakan Hadi Kadioglu and Ismail Hakki Aydin, in a paper by the above title, describe benefits of agmatine as relating to diffuse brain injuries.

Their study evaluates the effects of agmatine on histopathological damage following traumatic injury using a clinically relevant model of diffuse brain injury. A total of 27 male Sprague-Dawley rats weighing 200-225 g were anaesthetised and subjected to head trauma using Marmarou's impact-acceleration model. The rats were then separated into two main groups: one was treated with agmatine and the other with saline for up to 4 days immediately after head trauma. Rats from both groups were killed 1, 3 or 8 days post-injury. The brains were examined histopathologically and scored according to the axonal, neuronal and vascular changes associated with diffuse brain injury. There were no significant differences between the groups at 1 day or 3 days after trauma, but evaluation after 8 days revealed a significant improvement in the group treated with agmatine. The research data indicate that agmatine has a beneficial effect in diffuse brain injury and should be trialled for therapeutic use in the management of this condition.

Patients with severe head injuries may suffer from widespread brain damage secondary to trauma in the absence of a focal mass lesion that is not a consequence of herniation or perfusion failure. This type of diffuse brain injury is regarded as the most common cause of vegetative state and severe disability after closed head injury. It is termed diffuse axonal injury (DAI) in recognition of its pathologic nature. DAI is defined as the scattered destruction of axons throughout the brains of animals and humans that have sustained traumatic brain injury, typically involving acceleration or deceleration of the head. Agmatine is a putative neurotransmitter synthesised in the brain, stored in synaptic vesicles and released after membrane depolarisation. Apart from blocking Nmethyl-D-aspartate (NMDA) receptors, agmatine inhibits nitric oxide synthase (NOS) and induces the release of some peptide hormones. Agmatine exerts a neuroprotective effect by reducing the size of ischemic infarcts after focal or global ischemia in vivo. Agmatine also attenuates the extent of neuronal loss following spinal cord injury and helps prevent neurotoxicity. This study investigates whether agmatine attenuates diffuse traumatic brain injury histopathologically.

Results: Macroscopic Changes:

On gross pathological observation, the brains looked normal, with no contusion or focal lesions apart from mild subarachnoid haemorrhage in the basal cisterns.

Results: Microscopic Changes: Axonal changes:

Diffuse axonal swelling was less severe in the agmatine treatment group at days 1 and 3 post-injury. At day 8 post-injury, there was less axonal swelling in the agmatine group (FIG. 1).

Results: Microscopic Changes: Neuronal changes:

Pink shrunken neurons associated with perineuronal vacuolation were observed in both groups at days 1 and 3 post-injury. At day 8 post-injury, neuronal injury was less severe in the agmatine treatment group compared with the saline treatment group (FIG. 2).

Results: Microscopic Changes: Microvascular changes:

Microvascular changes, brain oedema and vascular congestion were more extensive in the saline treatment group than in the agmatine treatment group (FIG. 3). At days 1 and 3 post-injury, there was no statistically significant difference between the histopathological scores of the two groups (p=0.127, p=0.096, respectively). Evaluation at day 8, however, showed a statistically significant improvement in the agmatine group (p=0.04) (FIG. 4).

(7) The Multifaceted Effects of Agmatine on Functional Recovery after Spinal Cord Injury Through Modulations of BMP-2/4/7 Expressions in Neurons and Glial Cells

Yu Mi Park Won Taek Lee, Kiran Kumar Bokara, Su Kyoung Seo, Seung Hwa Park and Jae Hwan Kim, a team of American and Korean researchers, conducted a study reported under the foregoing title. Few treatments for spinal cord injury (SCI) are available and none have facilitated neural regeneration and/or significant functional improvement. Agmatine (Agm), a guanidinium compound formed from decarboxylation of L-arginine by arginine decarboxylase, is a neurotransmitter/neuromodulator and been reported to exert neuroprotective effects in central nervous system injury models including SCI. The purpose of their study was to demonstrate the multifaceted effects of Agm on functional recovery and remyelinating events following SCI. Compression SCI in mice was produced by placing a 15 g/mm2 weight for 1 min at thoracic vertebra (Th) 9 segment. Mice that received an intraperitoneal (i.p.) injection of Agm (100 mg/kg/day) within 1 hour after SCI until 35 days showed improvement in locomotor recovery and bladder function. Emphasis was made on the analysis of remyelination events, neuronal cell preservation and ablation of glial scar area following SCI. Agm treatment significantly inhibited the demyelination events, neuronal loss and glial scar around the lesion site. In light of recent findings that expressions of bone morphogenetic proteins (BMPs) are modulated in the neuronal and glial cell population after SCI, we hypothesized whether Agm could modulate BMP-2/4/7 expressions in neurons, astrocytes, oligodendrocytes and play key role in promoting the neuronal and glial cell survival in the injured spinal cord. The results from computer assisted stereological toolbox analysis (CAST) demonstrate that Agm treatment dramatically increased BMP-2/7 expressions in neurons and oligodendrocytes. On the other hand, BMP-4 expressions were significantly decreased in astrocytes and oligodendrocytes around the lesion site. Together, our results reveal that Agm treatment improved neurological and histological outcomes, induced oligodendrogenesis, protected neurons, and decreased glial scar formation through modulating the BMP-2/4/7 expressions following SCI.

Spinal cord injury (SCI) often results in permanent disability or loss of movement (paralysis) and sensation below the site of injury leading either to paraplegia (thoracic level injury) or tetraplegia (cervical level injury). SCI rostral to the lumbosacral level disrupts voluntary and supraspinal control of voiding and induces a considerable reorganization of the micturition reflex pathway. The urinary bladder is initially are flexic, but then becomes hyperreflexic because of the emergence of spinal micturition reflex pathway following SCI. SCI leads to neuronal and glial cell death, induces glial scar formation and inhibits axonal regeneration and remyelination. Oligodendrocytes produce myelin that wraps around the axons of neurons to enable them to conduct electrical impulses and neurotrophic factors to support the maintenance of nerve cells. Oligodendrocytes are lost during SCI, resulting in the loss of myelin and motor function that cause paralysis in animals. Agmatine (Agm) (4-aminobutyl guanidine), NH2-CH2-CH2-CH2-CH2-NH—C(—NH2) (═NH), is an endogenous amine and four carbon guanidine compound formed by decarboxylation of arginine. Agm was implicated in modulation of neurotransmission functions. It interacts with various neurotransmitter receptors, including nicotine, N-methyl-d-aspartate (NMDA), alpha2-adrenoceptors and imidazoline receptors. In addition, this molecule can interfere with second messenger pathways by acting as an adenosine diphosphate (ADP)-ribose acceptor thereby inhibiting ADP-ribosylation of proteins. Exogenous administration of Agm significantly reduces pain induced by inflammation following SCI. The above characteristics of Agm led the investigators to hypothesize that it might serve as a neuroprotective agent following neurotrauma. Bone morphogenetic proteins (BMPs) are multifunctional growth factors that belong to the transforming growth factor-b (TGF-b) super family. BMPs signal through serine/threonine kinase receptors, composed of type I and II BMP receptors. BMPs play important roles as trophic factors that may act in cell death regulation/differentiation, proliferation of neural progenitor cells and are also involved in restoration of injured neurons following various central nervous system (CNS) injuries. Among the various types of BMPs, BMP-2/7 in particular promotes differentiation and boosts dendrite growth in cultured striatal neurons and modulates the balance between glial cells and neurons. Earlier reports suggested that the BMP levels are altered following SCI. BMP-7 given intravenously showed neuroprotective effects following SCI. Furthermore, BMP-4 signaling was reported to be essential for astrocytes lineage proliferation following SCI. Conversely, disruption of BMP signaling in vivo negatively affects astrogliogenesis. Several groups have studied the effects of BMP signaling after SCI with mixed results. It was also demonstrated that BMP signaling enhances axonal outgrowth and locomotor recovery after SCI. These observations suggest that BMP signaling may be involved in both the beneficial and the detrimental effects following SCI. Agm treatment following SCI was shown to improve locomotor functions and reduce collagen scar formation accompanied with TGF-b and BMP-7 expressions suggesting that BMPs may regulate neural cell lineage commitment in vivo. Based on the previous evidences reporting the beneficial effects of Agm, it was hypothesized that Agm treatment, a well-known neuroprotector, may have effects on (1) recovery of locomotory and physiological functions, (2) facilitate axonal remyelination, (3) promote protection of neurons, (4) attenuate glial scar formation, and (5) modulate the BMP-2/4/7 expressions in neuronal and glial cells following SCI. In this study, the mice subjected to SCI were divided into agmatine treatment group (Agm treated group) and saline treatment group (EC group) along with parallel controls. All the experimental groups were examined for functional recovery and urinary bladder functions which included open field test and urine residual volume measurement respectively following SCI. Histological sections were examined to measure glial scar using an imaging program and the axonal remyelination was confirmed with myelin basic proteins (MBPs) staining. The surviving neurons, oligodendrocytes, and astrocytes were confirmed by counting the total cell numbers of microtubule-associated protein-2 (MAP-2), oligodendrocyte transcription factor-2 (Olig-2), and glial fibrillary acidic protein (GFAP) immunopositive cells in the total spinal cord (Th 8-Th 10 segments) and also in the rostral (Th 8), lesion (Th 9) and caudal regions (Th 10) separately using computer assisted sterological toolbox analysis (CAST) following SCI. This first study provides robust evidence of the beneficial effects of Agm treatment leading to lasting improvements of structure and function through modulating the BMP-2/4/7 expressions in neurons, oligodendrocytes, and astrocytes, which could be vital for directing the axonal remyelination and protect damaged neurons following SCI.

Citation: Park Y M, Lee W T, Bokara K K, Seo S K, Park S H, et al. (2013) The Multifaceted Effects of Agmatine on Functional Recovery after Spinal Cord Injury through Modulations of BMP-2/4/7 Expressions in Neurons and Glial Cells. PLoS ONE 8(1): e53911. doi:10.1371/journal.pone.0053911.

Results: Agmatine Treatment Enhanced Functional Outcome and Prevented Cell Death Following SCI:

The functional recovery of the mice subjected to SCI was assessed using the Basso Mouse Scale (BMS) scores. After SCI, the EC group (n=30) and Agm treated group (n=30) showed no ankle movement at 1 DPI (BMS score 0). Subsequently, the Agm treated group showed extensive ankle movements (BMS score 2) at 7 DPI and reached frequent or consistent plantar stepping, paws parallel at initial contact and lift off, and severe trunk instability (BMS score 6.8) at 21 DPI. At 35 DPI, the Agm treated group demonstrated frequent or consistent plantar stepping, some or mostly coordinated, paws parallel at initial contact and lift off, and normal trunk stability with up and down tail movement (BMS score 8). Even though the EC group showed occasional plantar stepping but the mice failed to frequent or consistent plantar stepping and their BMS scores were reached only up to a maximum of 4 points at 35 DPI. The behavioral test results suggested that Agm treated mice showed improvement in the functional recovery compared with that of the saline treated mice (FIG. 1). SCI results in the blockage of the reflex signals between the brain and the bladder and consequently, the self urination fails. Regaining bladder, bowel, and sexual function are some of the highest priorities following SCI and urinary function should be monitored as an outcome measure in SCI models. This study assessed whether Agm treatment could improve the bladder function following SCI (n=10, per group). Residual urine was collected and the volumes were measured until 14 DPI. Assessment of bladder function revealed that Agm treatment significantly reduced peak residual urine volumes from 4 day until 10 days compared to saline treated group. This effect of low residual volumes of urine was observed both in the EC and Agm treatment group after 10 DPI and the EC group showed improvement in self voiding and at the end of 14 DPI, both the Agm treated and EC group totally regained the normal voiding function with no urine residual volumes while manual pressing of the bladders. These results depict that Agm treatment could retain the balance between the brain and spinal cord and helps in maintaining the reflex signals for self-urination. This effect was not due to altered fluid intake because fluid consumption did not differ between Agm and saline treated groups (Data not shown). SCI induces a series of endogenous biochemical changes that lead to msecondary degeneration, including apoptosis. The p53-mediated apoptosis is likely to be an important mechanism of cell death in SCI. Our immunohistochemical staining results revealed that SCI induced the activation of p53 expression following SCI in the EC group. But fewer numbers of p53+ cells were seen in the lesion site in the Agm treated group compared with EC group both at 14 and 35 days DPI (Fig. S1).

Results: Agmatine Treatment Promoted Remyelination Following SCI:

SCI induces local inflammation and demyelination of the white matter around the lesion site, resulting in disrupted axonal conduction. Enhancement of oligodendrocytes or progenitors proliferation to enhance remyelination and functional recovery may lead to repair and restoration of locomotory function after SCI. To ascertain whether Agm could enhance the remyelination process following SCI, transmission electron microscopic studies (TEM) were performed to assess the micro-structural changes of the myelin sheath after SCI. TEM results suggested that Agm treated group (n=3) showed better pattern of myelination in the white matter of lesioned spinal cords compared to EC group (n=3) at 14 days following SCI. In the EC group, a number of large swollen axons with broken myelin sheaths were found across the degenerative white matter in the lesioned area. Thick myelin sheaths broke down and compact layers split apart irregularly and demyelinated axons were amongst the degenerative axons with enlarged spaces between the axolemma and deteriorating sheaths were also prominent in the EC group. Contrast to EC group, Agm treated group showed less broken myelin sheaths and more compact myelination around the lesion site. Moreover, demyelinated axons were found less across the degenerating area in the Agm treated group (FIG. 2A). The myelination was also checked by luxol fast blue staining in NC group, EC group and Agm treated group (n=3, per group) at 14 and 35 DPI. The staining results showed a remarkable increase of myelin (blue) and neurons (violet) stained cells indicating the reconstruction of lost myelin and neurons in the Agm treated group compared with the EC group (FIG. 2B). Following SCI, the fibers (serotonergic) originated from brain stem which extend the projections to the spinal cord get completely damaged at the lesion site which results in sensorimotor dysfunctions. To determine the effect of Agm (n=3) on regeneration of serotonergic fibers, which are important for the motor functional recovery of hind limbs, 5-HT immunostaining was performed in the distal segment of the spinal cord at 14 and 35 DPI. The staining results showed bead like structures with broken morphology of the serotogenic nerve fibers both at 14 and 35 DPI in the EC group (n=3). Conversely Agm treated group showed dense network of serotogenic fibers both at 14 and 35 DPI (Fig. S2) representing the restoration of sensorimotor dysfunctions.

These remyelination effects were further confirmed by checking the Olig-2 (oligodendrocyte marker) expression by western blotting. Western blot analysis showed that the Olig-2 proteinexpression in Agm treated group (n=5) was increased at all the time periods (from 1 DPI to 35 DPI) after compression SCI compared with that of the EC group (n=5) and the values reached significance at 35 DPI (FIG. 3A). Furthermore the number of Olig-2+ cells were counted in the EC group (n=5) and Agm treated group (n=5) using CAST analysis program. The CAST counting results showed that the numbers of Olig-2+ cells were higher in the total spinal cord (Th 8-Th 10 segments) at 1, 7, 14 and 35 DPI and significant increase was recorded at 14 and 35 DPI (FIG. 3B). There was an increase in the intensification of Olig-2+ cells in the rostral (Th 8), the lesion (Th 9), and the caudal segments (Th 10) of the injured spinal cord in the Agm treated group at 1, 7, 14, and 35 DPI and the significant difference between the groups (Agm treated group vs EC group) were recorded at 35 DPI (Fig. S4). Furthermore, remyelination (within the perimeter of the lesion site) in the injured spinal cord was confirmed by double immunostaining with Olig-2 (green) and MBP (red) at 35 DPI (FIG. 3C). Confocal microscopy results showed higher intensity of Olig-2+/MBP+ cells (rings formation indicated by a white arrow in FIG. 3C) in the Agm treated group (n=5) compared with the EC group (n=5). Considering that endogenous oligodendrocyte progenitorcells (NG2+) local to the lesion site are to be the source of new myelinating cells, experiments were done to check the expression of NG2+ cells in Olig-2+ and GFAP+ cell population at 7 and 35 DPI. Our immunohistochemical staining results showed that the expansion of NG2+/Olig-2+ cells outnumbered in the Agm treatment group both at 7 days and 35 DPI compared to ECgroup. But the NG2+/GFAP+ cells were less in Agm treated group both at 7 and 35 days DPI compared to EC group (Fig. S3). These results depict that Agm treatment could promote the formation of myelin sheath and might facilitate the remyelination process following SCI.

Results: Agmatine Treatment Protects the Damaged Neurons Following SCI:

Approaches to treat SCI include prevention of damaged neurons and regeneration of tissue loss. Strategies aimed to prevent neuronal damage will arise from secondary injury processes providing some hope for tissue sparing and improved functional outcome. To ascertain this, quantitative measurement of MAP-2 protein expression was done using western blot analysis after SCI. The densitometry results showed that the MAP-2 expression in the Agm treated group (n=5) was increased at 1, 7, 14 and 35 DPI (n=5) and the values reached significance at 14 and 35 DPI compared with the EC group (FIG. 4A). CAST analysis showed that the expansion of total number of surviving neurons (immunostained with MAP-2 antibody) in the total spinal cord (Th 8-Th 10 segments) (FIG. 4B) and in the rostral (Th 8), the lesion (Th 9) and the caudal (Th 10) segments of the spinal cord were increased in Agm treated group at all the time intervals compared with EC group (n=15, per group) (Fig. S5) and the increase was significant at 14 and 35 DPI compared with the EC group in the total spinal cord and in the rostral and caudal segments (n=15, per group). These results suggest that Agm treatment rescue the damaged neurons and accelerate the regeneration of damaged neurons after SCI. The neuronal nucleus (NeuN) and neurofilament (NF) expressions were found almost exclusively in neuronal cells and support the cytoskeleton following SCI. In our study immunofluorescence staining was done to detect the expressions of NeuN+/NF+ cells in EC and Agm treated groups. Immunofluorescence results showed higher number of NeuN+/NF+ cells in Agm treated group (n=5) compared with the EC group (n=5) and it seems that Agm treatment preserved the formation of dendrites and cell bodies of neurons around the lesion site in the injured spinal cord at 35 DPI (FIG. 4C). These findings suggest that Agm treatment attenuate the neuronal damage and aid for the neuronal survival following SCI.

Results: Agmatine Treatment Attenuated Glial Scar Formation and Reactive Gliosis at the Injury Site Following SCI:

SCI often results in permanent neurological impairment and axonal regeneration is made difficult due to astrocytes activation, oxidative stress, inflammation, cell death, and axon disruption. Recently it was reported that Agm treatment could support neuroregeneration by reducing the collagen scar area by decreasing the expression of TGFβ-2 and increasing the expression of BMP-7 following. In this study, the western blot results demonstrated significant decrease in the GFAP protein expression in the Agm treated group (n=5) at all the time periods (1, 7, 14, and 35 DPI) and the decrease was statistically significant at 14 DPI (FIG. 5A) compared with EC group (n=5). The number of GFAP+ cells were counted using CAST analysis from total spinal cord (Th 8-Th 10 segments) and at the rostral (Th 8), the lesion (Th 9), and the caudal (Th 10) segments of the spinal cord in EC and Agm treated groups (n=5, per group). The CAST analysis revealed significant decrease in the total number of GFAP+ cells in the total spinal cord and also in the rostral, lesion and caudal segments of the spinal cord in Agm treated group compared with the EC group at 7, 14, and 35 DPI. (FIG. 5B, S6). And also, the glial scar formation after SCI was determined by GFAP immunoreactivity at the injured site. The GFAP immunoreactivity was measured using the image analysis program. The results showed that Agm treatment significantly decreased the GFAP immunopositive area (n=5) compared with the EC group (n=5) at 7, 14 and 35 DPI and the decrease was found to be statistically significant at 14 and 35 DPI (FIG. 5C) suggesting that Agm treatment significantly attenuated the glial scar formation at 14 days following SCI (w, indicate glial scar area FIG. 5D).

Results: Agmatine Treatment Prevented the Neuronal & Oligodendrocytes Cell Loss and Attenuated the Astrocytes Formation Around the Lesion Site Following SCI:

Earlier it was reported that in the intact adult spinal cord the glial progenitor cells occupy 80% of the total cells. However, there is a substantial net increase in the progeny of damaged ependymal and astrocytes lineage cells following SCI. This study first aimed to count the total cell numbers of surviving neurons, astrocytes and oligodendrocytes in the NC, EC and Agmtreated groups (n=5, per group) in Th 8-Th 10 segments of the spinal cord using CAST analysis program (cells were read as actual number; 6106 cells/mm3) at 1, 7, 14 and 35 DPI using MAP-2, GFAP and Olig-2 antibodies. The CAST results showed that in the control mice (NC group) the average cell number representing neurons were 5.74260.274 (6106 cells/mm3). After SCI the total neuronal cell numbers were decreased in the EC group. However, Agm treatment dramatically attenuated the total neuronal cell loss following SCI and the average cell count number were significantly higher at all the time points compared with the EC group and the average numbers compared with EC group (EC vs Agm) were: 2.87560.043 vs 3.26860.065 at 1 day, 3.12760.065 vs 3.74560.056 at 7 days, 3.35960.095 vs 4.25160.109 at 14 days and 3.51760.144 vs 4.62260.273 (6106 cells/mm3) at 35 DPI. Specifically, the average numbers of surviving neurons in the Agm treated group were recorded to be almost similar to that NC group at 35 DPI. The total number of oligodendrocytes were also counted using CAST analysis. The CAST counting results revealed that the average Olig-2+ cell numbers in the NC group were 14.07862.037 (6106 cells/mm3). SCI resulted in the significant loss of oligodendrocytes. But, and the average cell count number from the CAST results showed 4.01860.030, 4.99760.205, 5.99360.082 and 9.01860.206 (6106 cells/mm3) at 1, 7, 14 and 35 DPI respectively in EC group. But, Agm treatment prevented the oligodendrocytes cell loss and the average Olig-2+ cells were 5.40460.008, 6.96460.04, 8.03060.112 & 12.00460.431 (6106 cells/mm3) at 1, 7, 14 and 35 DPI and the average cell numbers were found to be significant at 7 and 14 days compared to the EC group. The total numbers of GFAP+ cells were counted in the NC, EC and Agm treated groups. CAST analysis showed that in the NC group the average total cell numbers of GFAP+ cells were 2.09260.281 (6106 cells/mm3). Following SCI the total number of GFAP+ cells were decreased in the Agm treated group compared to the EC group and the decrease between EC vs Agm were: 1.70260.007 vs 1.50260.005 at 1 day, 3.50260.171 vs 2.20560.108 at 7 days, 3.70160.148 vs 2.39560.086 at 14 days and 3.75260.086 vs 2.50360.055 (6106 cells/mm3) at 35 DPI. The overall CAST results suggest that Agm treatment significantly prevented the neurons and oligodendrocytes cell loss and inhibited the formation of astrocytes. Moreover, the average cell numbers of all the cell types (neurons, oligodendrocytes and astrocytes) in Agm treated group at 35 DPI were almost reached to the normal control group (Table 1).

Results: Agmatine Treatment Modulated the BMP-2/4/7 Expressions Following SCI:

Bone morphogenetic proteins (BMPs) play a critical role in regulating cell fate determination during central nervous system (CNS) development and BMP-2/4/7 expressions in particularmodulates cell differentiation at the injury site following SCI. Taking into consideration the important roles of BMP-2/4/7 expressions following SCI, here, the authors intended to investigate whether Agm treatment could modulate the BMP-2/4/7 protein expressions and contribute for neurological recovery following SCI. The quantitative western blot results showed that the BMP-2/7 protein expressions were increased at 1, 7, 14, and 35 DPI and the values reached significant at 1 & 7 DPI and 7 & 14 DPI respectively in Agm treated group (n=5) compared with the EC group (n=5) (FIG. 6A, 6B). Conversely, the quantitative results of the BMP-4 protein expression was decreased at 1, 7, 14, and 35 DPI and the values were significant at 7, 14 and 35 DPI in the Agm treated group (n=5) compared with those of the EC group (n=5) (FIG. 6C).

Results: Agmatine Treatment Modulates the Expansion of Oligodendrocytes Progenitor Cells (NG2+) Via BMP-2/4/7 Expressions Following SCI:

Endogenous oligodendrocyte progenitor cells (NG2) local to the lesion site differentiate into oligodendrocytes and are responsible for myelin repair [41,42]. The expression pattern of NG2+ in BMP-2/4/7+ cell populations was determined by immunofluorescence staining. The results suggested that the NG2+/BMP-2+ cells were higher in the Agm treated group compared with EC group at 7 and 35 DPI. However, the NG2+/BMP-4+ cell population weredecreased at 7 DPI and the NG2+/BMP-4+ cells were almostdisappeared in the Agm group at 35 DPI. Conversely the expansionof NG2+/BMP-7+ cells was increased around the lesion site in Agm treated group at 35 DPI compared to EC group (n=5) (Fig. S8).

Results: Agmatine Treatment Increased the BMP-2/7 Expressions in Neurons and Oligodendrocytes and Decreased the BMP-4 Expressions in Astrocytes and Oligodendrocytes Following SCI:

BMPs are known to regulate proliferation or differentiation of neurons, oligodendrocytes, and astrocytes during CNS development. These authors hypothesized whether Agm treatment could modulate BMPs expression in neurons, astrocytes and oligodendrocytes after SCI. Recent findings demonstrated that BMPs show potential relationship with neurons and glial cells in the normal/injured spinal cord. In this study immunofluorescence and DAB staining (CAST analysis) were performed to co-localize and count the BMP-2/4/7+ cells in neurons, oligodendrocytes and astrocytes at 1, 7, 14 and 35 DPI. The immunofluorescence staining results showed higher number of BMP-2+/MAP-2+& BMP-2+/Olig-2+ cells in Agm treated group compared to the EC group at 7 and 14 days respectively following SCI (FIG. 7A). Similarly, CAST results showed the total number of BMP-2+/MAP-2+ and BMP-2+/Olig-2+ cells were higher in the Agm treated group (n=5) compared with the EC group (n=5) at 1, 7, 14 and 35 DPI and the increase in numbers were significant at 7 & 35 DPI and 7 & 14 DPI respectively in the Agm treated group (n=5) compared with EC group (n=5) (FIG. 8A, 8B). The average cell numbers representing the BMP-2 co-localized cells with MAP-2 and Olig-2 expressions were provided in the Table S2. It was reported that BMP-7 has been shown to exert neuroprotective effect after traumatic SCI and promote the functional recovery after contusion SCI. Here, they investigated whether Agm could modulate the BMP-7 expressions in neurons and oligodendrocytes after SCI. Dual immunofluorescence staining was performed to localize the BMP-7+/MAP-2+ and BMP-7+/Olig-2+ cells following SCI. The immunofluorescence staining results showed increased number of BMP-7+/MAP-2+ and BMP-7+/Olig-2+ cells at 7 and 14 DPI in the Agm treated group (n=4) compared with the respective EC group (n=4) (FIG. 7B). DAB immunostaining was also performed to localize the BMP-7 expression in neurons and oligodendrocytes and the total number of BMP-7+/MAP-2+ and BMP-7+/Olig-2+ cells were counted using the CAST analysis program. The CAST data showed that the number of BMP-7+/MAP-2+ and BMP-7+/Olig-2+ cells were increased in the Agm treated group (n=5) compared with the EC group (n=5) at 1, 7, 14, and 35 DPI and the values were significant at 7 & 14 DPI and 7 DPI respectively compared with that of the EC group (FIG. 8C, 8D). The average cell numbers representing the BMP-7 co-localized cells with MAP-2 and Olig-2 expressions was provided in the Table S2. BMP-4 can take part in inhibiting oligodendrocytes specification and differentiation to promote astrocytes proliferation after injury [16]. Previous findings suggest that BMP-4 increases reactive gliosis and glial scar formation at the lesion site following SCI. To ascertain the involvement of BMP-4 expression in modulating gliosis, immmunofluorescence staining was performed to localize the BMP-4+ cells in astrocytes and oligodendrocytes. Results showed that Agm treatment decreased the BMP-4+/GFAP+ cell population at 7 DPI and increased the BMP-4+/Olig-2+ cells expansion at 35 DPI (FIG. 7C). However, BMP-4 expression was not found to be co-localized with MAP-2 both in the EC and the Agm treated group (Fig. S7). Simultaneously, CAST analysis was performed to count the total number of BMP-4+ cells in astrocytes and oligodendrocytes in the EC (n=5) and Agm treated group (n=5) at 1, 7, 14, and 35 DPI. The CAST results showed that the total numbers of BMP-4+/GFAP+ and BMP-4+/Olig-2+ cells in the Agm treated group were decreased at all the time periods compared with the EC group and the decrease was found to be statistically significant at 7 & 14 DPI in astrocytes and at 14 & 35 DPI in oligodendrocytes (FIG. 8E, 8F) compared with EC group. The average cell numbers representing the BMP-4 co-localized cells with GFAP and Olig-2 expressions was provided in Table S2.

Supporting Information:

Their Figure S1 shows Agmatine treatment attenuated apoptosis following SCI. The EC (n=4) and Agm treated group (n=4) were immunostained with p53 antibody at (A) 14 and (B) 35 DPI. The p53 expression was substantially decreased after SCI in the Agm treated group compared with the EC group at 14 and 35 DPI. Scale bars: 50 mm. Their Figure S2 shows Agmatine treatment increased serotogenicfiber following SCI. Images were taken from the mice which received either Agm or saline (n=3, per group) following SCI. Agm treated mice showed dense network of 5-HT+ Serotonergic fibers in the caudal region of the spinal cord almost showing the same morphology to that of the normal control group (n=3). EC group showed the beaded and broken morphology of serotogenic fibers both at 14 and 35 DPI. Scale bars: 50 mm. Their Figure S3 shows Agmatine treatment increased the expansion of oligodendrocyte progenitor cells (NG2+) following SCI. Immunolocalization of NG2+ cells in astrocytes (GFAP+) and oligodendrocytes (Olig-2+) at (A) 7 and (B) 35 DPI. Thenumber of NG2+/GFAP+ cells were reduced in the Agm treated group (n=5) compared with the EC group (n=5) at (C) 7 & (D) 35 DPI. The NG2+/Olig-2+ cells expansion were outnumbered in the Agm treated group both at 7 & 35 DPI compared with EC group. Their Figure S4 Agmatine treatment increased the number of oligodendroyctes following SCI. The quantitative measurements of the total Olig-2+ cells by CAST analysis in (A) the rostral (Th 8), (B) lesion (Th 9) and (C) caudal (Th 10) regions after SCI. The results showed a significant increase of the Olig-2+ cells in Th 8, Th 9 and Th 10 segments of the injured spinal cord in the Agm treated group compared with the EC group and the values reached significance at 35 DPI (n=5). {, p<0.05 NC group vs EC group; #, p<0.05 NC group vs Agm treated group; *, p<0.05 EC group vs Agm treated group. Results represent mean+/−S.E.M. Their Figure S5 shows Agmatine treatment prevented neuronal cells death following SCI. The quantitative measurement of the total MAP-2+ cells using CAST analysis in (A) the rostral (Th 8), (B) the lesion (Th 9) and (C) the caudal (Th 10) regions of the injured spinal cord (n=5, per group). The results showed an increase of MAP-2+ cells in Th 8, Th 9, and Th 10 segments of the spinal cord in the Agm treated group (n=5) compared with the EC group (n=5) and significant increase was recorded at 14 and 35 DPI in rostral and caudal segments. {, p<0.05 NC group vs EC group; #, p<0.05 NC group vs Agm treated group; *, p<0.05 EC group vs Agm treated group. Results represent mean+/−S.E.M. Their Figure S6 shows Agmatine treatment reduced the number of astrocytes following SCI. The quantitative measurement of the GFAP+ cells using CAST analysis in the (A) rostral (Th 8), (B) lesion (Th 9) and (C) caudal (Th 10) regions of injured spinal cord. The results showed a significant decrease of the GFAP+ cells in Th 8, Th 9, and Th 10 segments of the injured spinal cord in the Agm treated group (n=5) compared with the EC group (n=5) at 7, 14, and 35 DPI. {, p<0.05 NC group vs EC group; #, p<0.05 NC group vs Agm treated group; *, p<0.05 EC group vs Agm treated group. Results represent mean+/−S.E.M. Their Figure S7 shows non co-localization of neurons and BMP-4 following SCI. There were no BMP-4 & MAP-2 co-localized cells both in the EC group (n=4) and Agm treated group (n=4) at (A) 7 days and (B) 35 days around the lesion site following SCI. Scale bars: in A, 100 mm & in B, 10 mm. Their Figure S8 shows Agmatine treatment increased oligodendrocyte progenitor cells (NG2+) following SCI. The expression of NG2+ in BMP-2/4/7+ cell population was determined by immunofluorescence staining. The NG2+/BMP-2+ cells were higher in the Agm treated group compared with EC group at (A) 7 and (B) 35 DPI. (C) Conversely the expansion of NG2+/BMP-7+ cells were increased around the lesion site in Agm treated group at 35 DPI compared to EC group. (D) NG2+/BMP-4+ cell population was decreased at 7 days and the (E) NG2+BMP-4+ cells were almost disappeared in the Agm treated group at 35 DPI.

(8) Beneficial Effect of Agmatine on Brain Apoptosis, Astrogliosis, and Edema after Rat Transient Cerebral Ischemia

A Tiawanese research team, Che-Chuan Wang, Chung-Ching Chiol, Ching-Hong Chang, Jinn-Rung Kuol and Ching-Ping Chang, in a paper of the foregoing title, reports as follows: Although agmatine therapy in a mouse model of transient focal cerebral ischemia is highly protective against neurological injury, the mechanisms underlying the protective effects of agmatine are not fully elucidated. This study aimed to investigate the effects of agmatine on brain apoptosis, astrogliosis and edema in the rats with transient cerebral ischemia. Following surgical induction of middle cerebral artery occlusion (MCAO) for 90 min, agmatine (100 mg/kg, i.p.) was injected 5 min after beginning of reperfusion and again once daily for the next 3 post-operative days. Four days after reperfusion, both motor and proprioception functions were assessed and then all rats were sacrificed for determination of brain infarct volume (2, 3, 5-triphenyltetrazolium chloride staining), apoptosis (TUNEL staining), edema (both cerebral water content and amounts of aquaporin-4 positive cells), gliosis (glial fibrillary acidic protein [GFAP]-positive cells), and neurotoxicity (inducible nitric oxide synthase [iNOS] expression).

Results:

The results showed that agmatine treatment was found to accelerate recovery of motor (from 55 degrees to 62 degrees) and proprioception (from 54% maximal possible effect to 10% maximal possible effect) deficits and to prevent brain infarction (from 370 mm3 to 50 mm3), gliosis (from 80 GFAP-positive cells to 30 GFAP-positive cells), edema (cerebral water contents decreased from 82.5% to 79.4%; AQP4 positive cells decreased from 140 to 84 per section), apoptosis (neuronal apoptotic cells decreased from 100 to 20 per section), and neurotoxicity (iNOS expression cells decreased from 64 to 7 per section) during MCAO ischemic injury in rats. The data suggest that agmatine may improve outcomes of transient cerebral ischemia in rats by reducing brain apoptosis, astrogliosis and edema.

Results: Agmatine Attenuates MCAO-Induced Motor Deficits:

Maximal grip angle 1-4 days after MCAO injury was significantly decreased for MCAO-injured animals treated with an i.p. dose of normal saline compared with MCAO sham controls (FIG. 1). The maximal grip angle 2-4 days after MCAO injury was significantly increased for MCAO-injured animals treated with an i.p. dose of agmatine compared with vehicle controls (62 degrees vs 54 degrees; FIG. 1).

Results: Agmatine Attenuates MCAO-Induced Proprioception Blockade:

The percentage of MPE or proprioception blockade 1-4 days after MCAO injury was significantly increased for MCAO-injured animals treated with an i.p. dose of normal saline compared with MCAO sham controls (FIG. 2). The MCAO-induced proprioception blockade 2-4 days after MCAO injury was significantly reversed by an i.p. dose of agmatine (100 mg/kg; p<0.05) (10% MPE vs 54% MPE; FIG. 2).

Results: Agmatine Attenuates MCAO-Induced Cerebral Infarction Volume:

The cerebral infarction volume 4 days after MCAO injury was significantly increased for MCAO-injured animals treated with an i.p. dose of normal saline compared with MCAO sham controls (FIG. 3). The infarction volume 4 days after MCAO injury was significantly reduced for MCAO-injured animals treated with an i.p. dose of agmatine compared with vehicle controls (370 mm3 vs 50 mm3) (FIG. 3).

Results: Agmatine Attenuates MCAO-Induced Cerebral Edema:

The brain water content 4 days after MCAO injury was significantly increased for MCAO-injured animals treated with an i.p. dose of normal saline compared with MCAO sham controls (FIG. 4). The cerebral water content 4 days after MCAO injury was significantly decreased for MCAO-injured animals treated with an i.p. dose of agmatine compared with vehicle controls (82.5% vs 79.4%; FIG. 4).

Results: Agmatine Attenuates MCAO-Induced Cerebral Gliosis:

The numbers of GFAP-positive cells 4 days after MCAO injury was significantly increased for MCAO-injured animals treated with an i.p. dose of normal saline compared with MCAO sham controls (FIG. 5). The increased numbers of GFAP-positive cells 4 days after MCAO injury was significantly reversed for MCAO-injured animals treated with an i.p. dose of agmatine compared with vehicle controls (100 GFAP-positive cells vs 20 GFAP-positive cells per section; FIG. 5).

Results: Agmatine Attenuates MCAO-Induced Overexpression of iNOS:

The numbers of iNOS-positive cells 4 days after MCAO injury was significantly increased for MCAO-injured animals treated with an i.p. dose of normal saline compared with MCAO sham controls (FIG. 6). The increased numbers of iNOS-positive cells 4 days after MCAO injury was significantly decreased for MCAOinjured animals treated with an i.p. dose of agmatine compared with vehicle controls (64 iNOS-positive cells vs 7 iNOS-positive cells per section; FIG. 6).

Results: Agmatine Attenuates MCAO-Induced Neuronal Apoptosis:

The numbers of neuronal apoptosis cells 4 days after MCAO injury was significantly decreased for MCAOinjured animals treated with an i.p. dose of normal saline compared with MCAO sham controls (FIG. 7). The decreased numbers of neuronal apoptosis cells 4 days after MCAO injury was significantly decreased for MCAO-injured animals treated with an i.p. dose of agmatine compared with vehicle controls (100 vs 20 cells per section; FIG. 7).

Results: Agmatine Attenuates MCAO-Induced Overexpression of AQP4:

The numbers of cerebral AQP4-positive cells 4 days after MCAO injury was significantly increased for MCAO-injured animals treated with an i.p. dose of normal saline compared with MCAO sham controls (FIG. 8). The increased numbers of cerebral AQP4-positive cells 4 days after MCAO injury was significantly decreased for MCAO-injured animals treated with an i. p. dose of agmatine compared with vehicle controls (140 vs 84 AQP4-positive cells per section; FIG. 8).

Conclusions:

In summary, these findings revealed that agmatine therapy reduced cerebral infarct volume by 87%, brain edema formation by 50% and functional deficits by 70% 4 days after transient cerebral ischemia in the rat. In addition, both cerebral gliosis (evidenced by overexpression of both GFAP and AQP4) and neuronal apoptosis (evidenced by increased numbers of NeuN plus TUNEL double positive cells) and neurotoxicity (evidenced by increased iNOS expression) that occurred during transient cerebral ischemia could be greatly attenuated by agmatine therapy. It is likely that agmatine therapy improved the neurological outcome after transient cerebral ischemia by reducing neuronal apoptosis, gliosis, neurotoxicity and cerebral edema formation in the rat.

(9) Antidepressant Like Effect of Selective Serotonin Reuptake Inhibitors Involve Modulation of Imidazoline Receptors by Agmatine

Brijesh G. Taksande, Nandkishor R. Kotagale, Sunil J. Tripathi, Rajesh R. Ugale, Chandrabhan T. Chopde, in a paper having the foregoing title, looked a agmatine and imidiazoline receptors in antidepressant like effect of selective serotonin reuptake inhibitors (SSRIs) and imipramine. Recent findings demonstrated the dysregulation of imidazoline receptor binding sites in major depression and their normalization by chronic treatment with antidepressants including selective serotonin reuptake inhibitors (SSRIs). Present study investigated the role of agmatine and imidazoline receptors in antidepressant like effect of SSRIs and imipramine in mouse forced swimming test (FST) paradigm. The antidepressant like effect of fluoxetine or paroxetine was potentiated by imidazoline I1/I2 receptor agonist agmatine (5-10 mg/kg, ip), imidazoline I1 receptor agonists, moxonidine (0.25-0.5 mg/kg, ip) and clonidine (0.015-0.03 mg/kg, ip), imidazoline I2 receptor agonist, 2-(2-benzofuranyl)-2-imidazoline (5-10 mg/kg, ip) as well as by the drugs known to increase endogenous agmatine levels in brain viz., L-arginine, an agmatine biosynthetic precursor (40 mg/mouse, icv), ornithine decarboxylase inhibitor, difluoromethyl ornithine (12.5 mg/mouse, icy), diamine oxidase inhibitor, aminoguanidine (6.5 mg/mouse, icy) and agmatinase inhibitor, arcaine (50 mg/mouse, icy). Conversely, prior administration of I1 receptor antagonist, efaroxan (1 mg/kg, ip), I2 receptor antagonist, idazoxan (0.25 mg/kg, ip) and arginine decarboxylase inhibitor, D-arginine (100 mg/kg, ip) blocked the antidepressant like effect of paroxetine (10 mg/kg, ip) and fluoxetine (20 mg/kg, ip). On the other hand, antidepressant like effect of imipramine was neither augmented nor attenuated by any of the above drugs. Mice pretreated with SSRIs but not imipramine and exposed to FST showed higher concentration of agmatine in brain as compared to saline control. This effect of SSRIs on agmatine levels was completely blocked by arginine decarboxylase inhibitor D-arginine but not by imidazoline receptor antagonists, efaroxan or idazoxan. These results demonstrate that modulation of imidazoline receptors by agmatine are implicated in the antidepressant like effect of SSRIs and may be projected as a potential therapeutic target for the treatment of depressive disorders.

One of the very significant aspects of the present invention is a method of delivery: via the nasal cavity and with force to penetrate the olfactory area. As mentioned, this results in rapid delivery to the blood and brain to maximize dose effectiveness (amount of actives that given the opportunity to work before dissipation and/or deterioration) and delivery effectiveness (reduction of time to arrive at effective location).

By combining the beneficial therapeutic and other effects of agmatine treatment and maximize delivery methods, an improved therapy is created. In this way, the beneficial effects of the agmatine, such as described significantly above, are combined with the beneficial effects of maximum efficientcy delivery methodology. Further, the liquid carrier for the agmatine moisturizes the nasal cavities and acts as a base host for the agmatine as it acts on and penetrates the nasal cavity walls and olfactory region. This combination of utilizing the nasal cavitity delivery under pressure, along with the broad benefits of agmatine results in an unexpected synergistic effect.

In addition to the beneficial agmatine and its delivery methods herein, as described above, in some preferred embodiments of the present invention, there is further included at least one additional active component. These active components may be any of one or more beneficial additions that are compatible with agmatine and have some medicinal, curative, pain relieving or moisturizing effect on the sinus cavity walls, vascular system upper respiratory system, olfactory region and/or brain. These include, but are not limited to, moisturizers, humectants, over the counter drugs and prescription drugs. Such drugs may be antihistamines, infection treatments, antioxidants, cell growth accelerators, anti-inflammatories, vasoconstrictors, nasal decongestants, or other nasal cavity, wall or upper respiratory treatments. Preferred actives are moisturizers, decongestants, antihistamines, infection treatments and anti-inflammatories. One interesting additive formulatrion would include salt to create a saline or other salt-based carrier. Effervescent saline could be utilized as a preferred carrier with other active(s) a. Carbon dioxide to the nasal cavitivy has numerous curative effectives and saline is well known for its reduction of swelling and other benefits to the nasal area. Another interesting addivitive formulation may involve the use of pyruvates and derivatives, with their well known upper respiratory, respiratory and other benefits. Examples of moisturizers and humectants are: glycerin, propylene glycols (MW 400 to 8000), maltodextrins (liquid), honey, pectin, hydroxypropyl methylcellulose, and carboxymethylcellulose. Examples of topical decongestants are: ephedrine, levomethamphetamine, naphazoline, oxymetazoline, phenylephrine, pseudoephedrine, tramazoline, and xylometazoline. The actives may also be fragrance sensations or fragrance with other benefits, such as eucalyptus, menthol or lavender.

The liquid carrier for the present invention treatments may be water, water in combination with other liquids, or non-aqueous solutions, provided that they are compatible with agmatine and appropriate for human nasal cavity use. Saline is a desirable carrier as it also provises a soothing and swell-reduction effect to the nasal cavity due to its beneficial osmotic and othe positive effects.

Referring now to the drawings, like reference numerals designate corresponding parts throughout the several views, various embodiments of the present invention are shown.

FIG. 1 is a block diagram of an embodiment of the present invention agmatine nasal delivery treatment method: agmatine and a liquid carrier, as well as optionally, with additional active component(s) in addition to agmatine, However, because the present invention includes treatments with and without other actives, the following discussions below should be taken to mean with or without additional active additives.

FIG. 1 illustrates an agmatine treatment method pursuant to the present invention. The term “treatment” as used herein should be interpreted broadly to mean preventive treatment as well as post-injury treatment, as well as during ailment treatment. As FIG. 1 shows, the non-inhaled dosage 1, contains agmatine (in a preferred dosage concentration discussed below), a liquid carrier, such as water, and optional (at least one) additional actives. The dosage may be a single dose or multiple doses in a container with a regulator, and the container may be pressurized or pump type dispensers. The non-inhaled therapeutic or medicinal dosage travels through a flow-regulating device 3. In preferred embodiments, the flow-regulating device 3 controls the flow rate 7 of the dosage 1 at a rate that is safe and comfortable for the patient or other user. In the embodiment shown in FIG. 1, the flow rate 7 of the therapeutic non-inhaled dosage is between 1 cubic centimeter per second (cc/sec) and 20 cc/sec. In preferred embodiments of the present invention shown in FIG. 1, the flow rate is adjustable to any value between 1 cc/sec and 20 cc/sec.

The therapeutic non-inhaled dosage 1 has a flow duration 9. The flow duration 9 is the length of time during which that the non-inhaled dosage flows through the flow regulating device into at least one nasal cavity 11 of a patient or other user. In the embodiment shown in FIG. 1, the flow duration 9 is shown as lasting between 2 and 30 seconds. In preferred embodiments of the present invention, the flow duration is adjustable to any value between 2 and 30 seconds. Typically, 2 to 5 seconds is preferred and multiple applications may be sprayed in a single sitting, depending upon the ultimate dosage required for the particular treatment.

After the therapeutic non-inhaled dosage 1 leaves the flow regulating device 3, it enters at least one nasal cavity 11 of a patient. The therapeutic non-inhaled dosage 1 is adsorbed by the nasal tissue and subsequently absorbed by the body. This adsorption and subsequent absorption can have a beneficial effect on many ailments, some of which are shown in FIG. 2. The pressurized delivery through the olfactory region and into the brain, provides for efficient and rapid delivery.

The additional step 5 of instructing the patient to refrain from inhaling protects the patient from accidently inhaling the dosage 1 into the lungs, thus delaying delivery to the brain and reducing efficacy while promoting dilution.

Turning now to FIG. 2, a block diagram, block 21, shows some of the medical conditions (ailments) that can be treated using the present invention agmatine solution nasal cavity delivery methods (with and without additional actives). In some embodiments of the present invention, the used for any one or more of the following: stroke; spinal cord injury; opiate use; cannabinoid use; depression; trauma to the brain. The present invention methods may be used preventatively, i.e., prior to the appearance or occurance of any of the foregoing medical conditions, or after the condition(s) occur, or both. For example, a football team may have all of its members take the herein prescribed agmatine solution treatments just before a game, and if a player has a concussion or other head or spinal cord injury, may again be treated with agmatine solution.

Turning now to FIG. 3, a block diagram, block 31, shows the durations of medicinal non-inhaled dosage used in some embodiments of the present invention nasal delivery methods and treatments. The durations listed in FIG. 3 are ranges, so the actual duration can be any value between the low end of the range and the high end of the range, inclusive. In some embodiments of the present invention, the duration 29 lasts between 2 and 30 seconds. In other embodiments of the present invention, the duration 31 lasts between 2 and 15 seconds. In still other embodiments of the present invention, the duration 33 lasts between 5 and 10 ten seconds. Durations of less than 2 seconds and more than 30 seconds are also considered to be within the scope of the invention.

Turning now to FIG. 4, another embodiment of the present invention agmatine nasal delivery methods and treatments is shown. FIG. 4 is a block diagram of an embodiment of the present invention nasal delivery methods and treatments that incorporates many aspects shown in FIG. 1, and identical blocks are identically numbered. Here, repeat dosages 13 are indicated.

The therapeutic non-inhaled dosage travels through a flow-regulating device 7. In preferred embodiments, the flow-regulating device 7 controls the flow rate 9 of the therapeutic non-inhaled dosage 1 at a rate that is safe and comfortable for the patient. In the embodiment shown in FIG. 1a, the flow rate 9 of the therapeutic non-inhaled dosage is between 1 cubic centimeter per second (cc/sec) and 20 cc/sec. In preferred embodiments of the present invention shown in FIG. 1a, the flow rate is adjustable to any value between 1 cc/sec and 20 cc/sec.

The therapeutic non-inhaled dosage 1 has a flow duration 11. The flow duration 11 is the length of time during which the therapeutic non-inhaled dosage flows through the flow regulating device into at least one nasal cavity 13 of a patient. In the embodiment shown in FIG. 1, the flow duration 11 is shown as lasting between 2 and 30 seconds. In preferred embodiments of the present invention, the flow duration is adjustable to any value between 2 and 30 seconds.

After the therapeutic non-inhaled dosage 1 leaves the flow regulating device 7, it enters at least one nasal cavity 13 of a patient. The therapeutic non-inhaled dosage 1 is adsorbed by the nasal tissue. This adsorption can have a beneficial effect on many head ailments, some of which are shown in FIG. 2. The additional step 5 of instructing the patient to refrain from inhaling protects the patient from accidently inhaling the therapeutic non-inhaled dosage 1.

In the embodiment shown in FIG. 4, identical steps to those of FIG. 1 are identically numbered and not fully repeated here. After the therapeutic non-inhaled dosage 1 passes through the flow regulating device 3 and into the at least one nasal cavity 11 of a patient, the dose is repeated 13. In some preferred embodiments, the dose is repeated 13 between one and ten times. In still other embodiments, the dose is repeated more than ten times. The step 13 of repeating the dose can be used if a single application of the non-inhaled dosage 1 is insufficient to alleviate the head ailment or other ailment from which the patient suffers.

Turning now to FIG. 5, a block diagram, block 51, shows flow rates used in some embodiments of the present invention agmatine nasal delivery methods and treatments. The flow rates used in FIG. 5 are shown as ranges, and the actual rate of the flow may any value between the low end of the range and the high end of the range, inclusive. In some embodiments, a rate 37 between 1 cc/sec and 20 cc/sec is used. In other embodiments, a flow rate 39 between 2 cc/sec and 10 cc/sec is used. In other preferred embodiments, a flow rate 41 between 1 cc/sec and 5 cc/sec is used. In still other preferred embodiments, a flow rate 43 between 4 cc/sec and 5 cc/sec is used. In still other preferred embodiments, a flow rate 45 of approximately 10 cc/sec is used. Embodiments with flow rates of less than 1 cc/sec or more than 20 cc/sec are also considered to be within the scope of the invention.

Turning now to FIG. 6, a block diagram, block 61, shows concentration levels of agmatine in the liquid carrier. In some preferred embodiments, the present invention agmatine solution contains about 2 to about 200 mg of agmatine per ml of liquid carrier. In more preferred embodiments, the agmatine solution contains about 4 to about 40 mg of agmatine per ml of liquid carrier. In the most preferred embodiments, the agmatine solution contains about 5 to about 15 mg of agmatine per ml of liquid carrier. One very good dosage concentration level for all uses stated above are these just stated ranges, and especially about 10 mg per liter. The dosages are dependent upon the weight of the patient, the metabolism and ailment treated. For long term treatments, such as spinal injury and depression, ongoing regimens over days, week or months may be beneficial, taken at least twice a day and having 5 to 10 “shots” per use, in the 2 to 5 second range.

FIG. 7 illustrates a block diagram showing nasal treatment delivery devices that may be used in the present invention methods. Here, block 71 illustrates the caption of the Figure, namely, nasal treatment delivery devices. Block 73 shows that the flow regulating device used in the present invention methods may be a single dose dispenser (monodose) with a pressure control valve for flow rate regulation. The rate of flow is set in accordance with the ranges set forth above. In the case of a monodose dispenser, the entire dose is dispensed, so that time of dispensing does not need to be controlled—it is just the controlled flow rate over time it takes to unload the dose. Thus, a monodose dispenser may controllably release a pressurized mixture of the agmatine and its liquid carrier, until it stops flowing. The various types of mechanisms for driving the contents from the container to the nasal cavity are also exemplified. These include squeeze mechanisms where the squeeze component or bulb is below the content so that external squeeze pressure forces out the content, much like a turkey baster; squeeze mechanisms where the squeeze component is the actual dose holding aspect of the container, like a nasal decongestant squeeze spray container; push mechanisms that physically operate much like syringes but may have more complex internal aspects, such as piercers or counter-biased valving; and others, referring to any known controlled flow mechanism available to the artisan.

On the other hand, a plural or multidose dispenser may be used, and needs dispensing on/off control, otherwise the entire contents could be unnecessarily released in one shot. Thus, block 75 illustrates the use of a multidose dispenser with a pressure control valve for flow rate regulation. The rate of flow is set in accordance with the ranges set forth above. Block 77 shows one multidose dispenser option wherein the user controls the release time, so that there is variable dosage. For example, there may be an activator, such as a push button or a squeeze mechanism to release the dosage, and the user may be directed to dispense for a time, e.g., dispense for eight to ten seconds. Alternatively, as shown in block 79, an auto-controlled release mechanism may be used, e.g., a spring return release that closes a valve based on set timing, or a dual spring device with one being reverse spring mechanism that returns a lever to control the time of release. Timed valving is well known in the field of medicine dispensing and any available multidose fixed time dispensing mechanism may be utilized.

In FIG. 7, block 73 shows the main housing and dosage. It contains a dosage of agmatine liquid according to parameters as more specifically set forth above. Block 79 shows that the main housing 73 may have two open ends or one open end. In the case of one open end, the top end would include the release control and dispenser head mechanisms, with a closed bottom. In the case of a main housing with two open ends, one end would have the release control and dispenser head mechanisms and the other end would contain a moveable drive mechanism such as a pressure release mechanism, a piercer or a plunger (drive piston). Block 81 shows that the main housing 73 may be at least partially flexible or it may be inflexible. If the driver is the squeezing of the main housing, it must be flexible. If the driver a moveable component attached to the main housing 71 (a push or squeeze mechanism), then the main housing 71 is preferably inflexible.

Block 83 shows the dosage release control component. Block 85 illustrates the options for the dosage release control component, which are: frangible, puncturable, one-way valve, or gate. Block 87 shows the dosage dispenser head, which Block 89 then shows the options for, which are: perforated, hard, soft, or delivery cover (sponge, foam, cotton batting, or other). Block 74 shows the optional nose guard flange for the agmatine delivery device 71.

FIG. 8 illustrates a front partially cut view of one embodiment of a present invention nasal cavity delivery/treatment device 90. It includes a main housing 91 with a top 93 having a hollow central area containing a dosage of the present invention medicine. This storage area may be the inside of the main housing, or it may be one or more subunits—compartments, capsules, tanks, pouches, etc, within the main housing.

In this embodiment, the main housing 91 has attached to its distal end a dosage control component that is a spray release nozzle 95 that is set for prescribed flow rates within the ranges set forth in the present invention claims and as described above. Internal bag container 105 contains the agmatine liquid solution of the present invention and external pressure on bag 105 is created by pressurized gas located in space 107 inside main housing 91. At top 93 is a dosage dispenser head, in this case, a push dispenser mechanism 97 that includes release orifice 101, actuation tube 99 and push pad 103. A user inserts push dispenser mechanism 97 into a nasal cavity at its distal end (orifice 101) while holding nasal treatment delivery device 90 and then presses push pad 103 to release the contents. The flow regulation is set to an acceptable range so as to be relatively gentle to the user. This may include ranges in the order of 1 cc/sec to 10 cc per second. Typically this is a multidose device wherein the user is given instructions to dispense for a specified time period while not breathing, e.g., three seconds at full depression per nostril twice a day as needed. Alternatively, a built-in timer could automatically control the dose. For example, the device could have a slow spring closure that would require reset and re-push to reactivate.

FIG. 9 shows an alternative nasal treatment delivery device 110. This is an insert and squeeze device that includes a main body 111 with flexible walls and a dispensing nozzle 115 at its top 113. There is a stop 117 and threads 109 and a tapered dispensing tip 119 designed for nasal cavity insertion. There is a flow control valve 112 that regulates the rate of delivery. Additional valving, such as a duck bill valve, may also be included. The present invention methods agmatine solution is contained within the main housing 111 and is dispensed by a user inserting and squeezing, preferably while holding his/her breath.

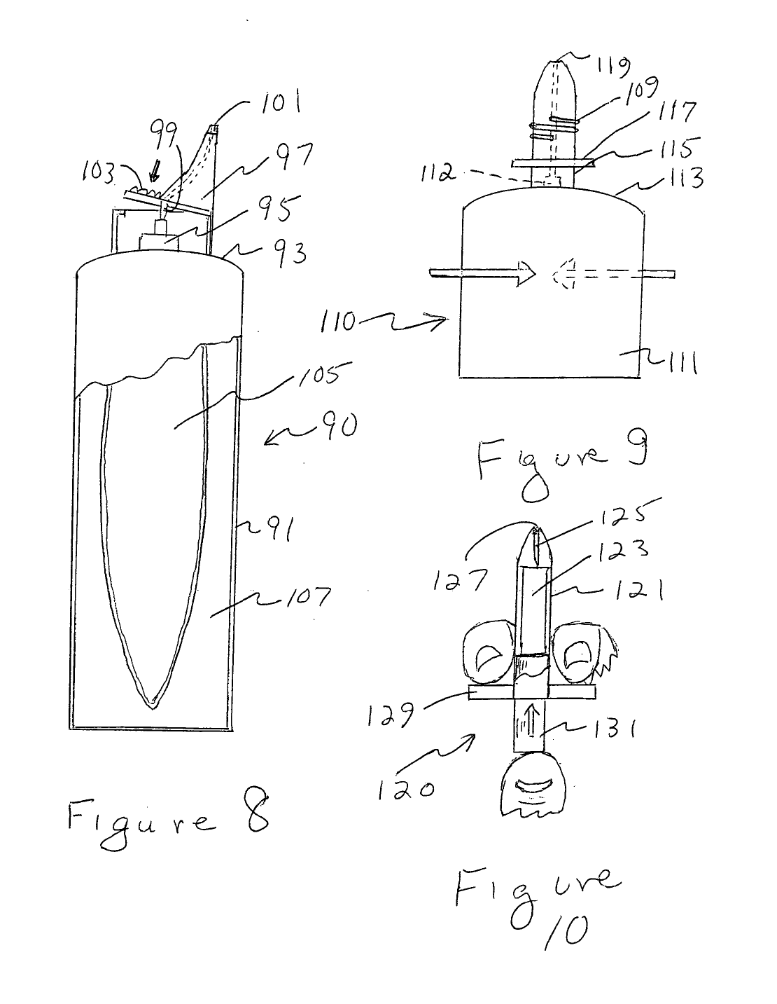

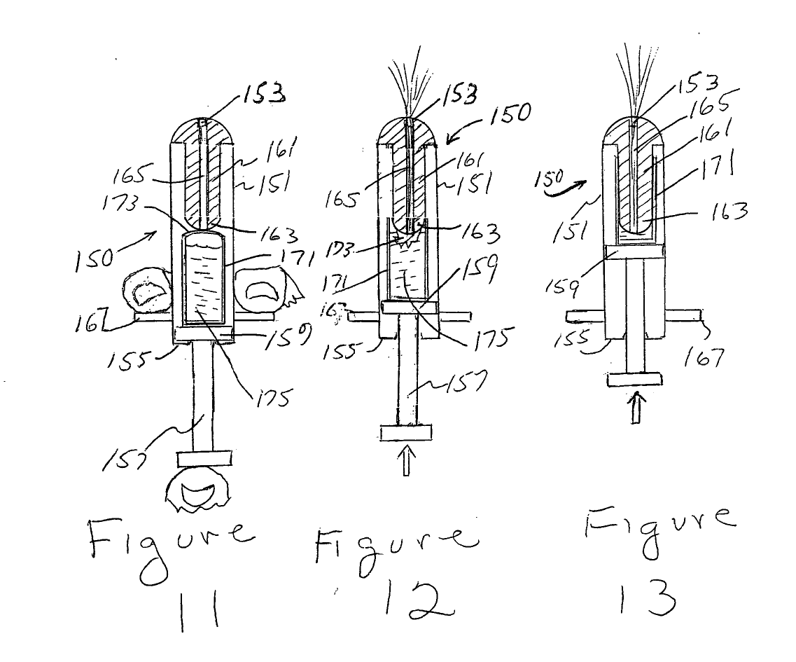

FIG. 10 shows a front partially cut view of a present invention nasal treatment delivery device 120 being held in a hand using two fingers and a thumb, as shown. There is a main housing 121 and a vertically moveable piston 131. A rigid, semi-flexible or flexible container or pouch 123 contains the agmatine solution of the present invention and piercing tube 125 is connected to flow control valve 127. A user holds nasal treatment delivery device 120 as shown, inserts it into a nasal cavity, and pushes piston 131 upwardly to force pouch 123 to rupture via piercing tube 125 for forced medicine release under pressure through valve 127 to the nasal cavity walls.

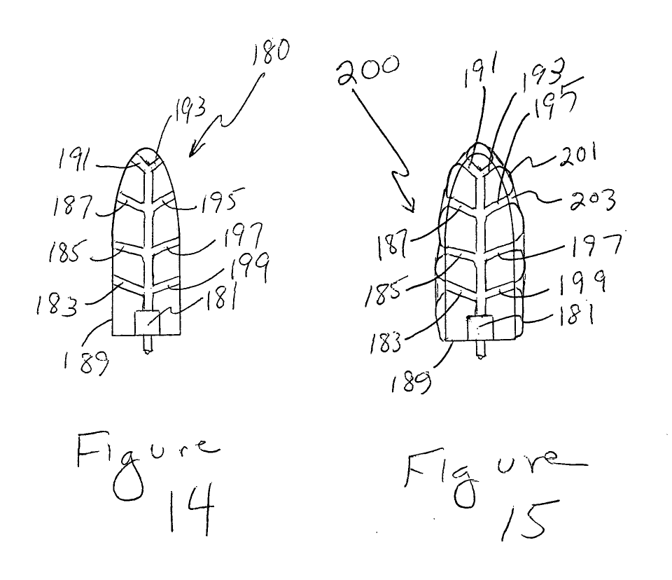

FIGS. 14 and 15 show alternative types of dosage dispenser heads that may be used in present invention device: one has multiple release ports and the other has multiple release ports with a soft contact sheath. FIG. 14 shows a cut front view of one dosage dispenser head 180 that may be used in conjunction with a present invention device. It includes a control valve 181 to regulate release of agmatine solution to be within the proscribed ranges set forth above. Upstream from control valve 181 is a main flow channel 183 with branches 185, 187, 189, 191,193 and 195 to show a diverse multiport manifold head for diverse. This dosage dispensing head will direct the gas/liquid medicine in many directions simultaneously to more evenly and quickly coat the sinus cavity wall.

FIG. 15 shows a similar present invention dosage dispensing head 200, but with a soft pad for nasal wall comfort. This pad does not cover the spay ports and may be made of soft pervious or impervious materials such as various foams or skins.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

1.-19. (canceled)

20. A method of delivering agmatine to a human for medicinal treatment, which comprises:
a) providing a spray dispenser with an agmatine solution containing agmatine in a liquid carrier, said spray dispenser having a dispensing nozzle;
b) positioning the dispensing nozzle of said spray dispenser adjacent a nasal cavity of said human and spraying an effective amount of said agmatine solution under pressure into said nasal cavity so as to penetrate an olfactory area of said human; and
wherein said medical treatment is for one of the group consisting of stroke ailment, spinal cord injury, depression ailment, traumatic brain injury, and adjunct treatment.

21. The method of delivering agmatine to a human for medical treatment of claim 20 wherein said liquid carrier is water.

22. The method of delivering agmatine to a human for medical treatment of claim 20 wherein said liquid carrier is a saline solution.

23. The method of delivering agmatine to a human for medical treatment of claim 20 wherein said medicinal treatment is medical treatment taken for said stroke ailment.

24. The method of delivering agmatine to a human for medical treatment of claim 20 wherein said medicinal treatment is medical treatment taken for said spinal cord injury.

25. The method of delivering agmatine to a human for medical treatment of claim 20 wherein said medicinal treatment is said adjunct treatment with opiate treatment for pain.

26. The method of delivering agmatine to a human for medical treatment of claim 20 wherein said medicinal treatment is said adjunct treatment with cannabinoid treatment for pain.

27. The method of delivering agmatine to a human for medical treatment of claim 20 wherein said medicinal treatment is for said traumatic brain injury.

28. The method of delivering agmatine to a human for medical treatment of claim 20 wherein said medicinal treatment is for said depression ailment.

29. The method of delivering agmatine to a human for medical treatment of claim 20 wherein said medicinal treatment is for said stroke ailment.

30. The method of delivering agmatine to a human for medical treatment of claim 20 wherein said spray dispenser is selected from the group consisting of a pressurized spray dispenser and a manual pumped dispenser.

31. The method of delivering agmatine to a human for medical treatment of claim 20 wherein said spray dispenser and said agmatine solution at a rate of 0.05 to 4.0 cc/sec.

32. The method of delivering agmatine to a human for medical treatment of claim 20 wherein said agmatine solution contains about 2 to about 200 mg of agmatine per ml of liquid carrier.

33. The method of delivering agmatine to a human for medical treatment of claim 20 wherein said agmatine solution contains about 4 to about 40 mg of agmatine per ml of liquid carrier.

34. The method of delivering agmatine to a human for medical treatment of claim 20 wherein said agmatine solution contains about 5 to about 15 mg of agmatine per ml of liquid carrier.
